(19) United States
(12) Patent Application Publication
(10) Pub. No.: US 2004/0248956 A1
(43) Pub. Date: Dec. 9, 2004

(54) Substituted imidazoles as cannabinoid receptor modulators

(76) Inventors: William K Hagmann, Westfield, NJ (US); Hongbo Qi, Edison, NJ (US); Shrenik K. Shah, Metuchen, NJ (US)

Correspondence Address: MERCK AND CO INC, P O BOX 2000, RAHWAY, NJ, 070650907

(21) Appl. No.: 10/501,060

(22) PCT Filed: Jan. 24, 2003
(86) PCT No.: PCT/US03/02351

Related U.S. Application Data
(60) Provisional application No. 60/352,743, filed on Jan. 29, 2002.

(51) Int. Cl. A61K 31/4178

(52) U.S. Cl. 514/396; 514/397

(57) ABSTRACT

Compounds of the present invention are antagonists and/or inverse agonists of the Cannabinoid-1 (CB1) receptor and are useful in the treatment, prevention and suppression of diseases mediated by the Cannabinoid-1 (CB1) receptor. The compounds of the present invention are useful as psychotropic drugs in the treatment of psychosis, memory deficits, cognitive disorders, migraine, neuropathy, neuro-inflammnatory disorders including multiple sclerosis and Guillain-Barr syndrome and the inflammatory sequelae of viral encephalitis, cerebral vascular accidents, and head trauma, anxiety disorders, stress, epilepsy, Parkinson's disease, movement disorders, schizophrenia. The compounds are also useful for the treatment of substance abuse disorders, the treatment of obesity or eating disorders, as well as, the treatment of asthma, constipation, chronic intestinal pseudo-obstruction, and cirrhosis of the liver. Particular novel compounds of structural formula (I) are also claimed.

### SUMMARY OF THE INVENTION

The present invention is concerned with substituted imidazoles of the general Formula I:

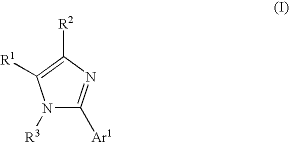

and pharmaceutically acceptable salts thereof which are antagonists and/or inverse agonists of the Cannabinoid-1 (CB1) receptor and are useful in the treatment, prevention and suppression of diseases mediated by the Cannabinoid-1 (CB1) receptor. The invention is concerned with the use of these novel compounds to selectively antagonize the Cannabinoid-1 (CB1) receptor. As such, compounds of the present invention are useful as psychotropic drugs in the treatment of psychosis, memory deficits, cognitive disorders, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, particularly to opiates, alcohol, marijuana, and nicotine, including smoking cessation. The compounds are also useful for the treatment of obesity or eating disorders associated with excessive food intake and complications associated therewith. The compounds are also useful for the treatment of constipation and chronic intestinal pseudo-obstruction. The compounds are also useful for the treatment of cirrhosis of the liver. The compounds are also useful for the treatment of asthma.

The present invention is also concerned with treatment of these conditions, and the use of compounds of the present invention for manufacture of a medicament useful in treating these conditions. The present invention is also concerned with treatment of these conditions through a combination of compounds of formula I and other currently available pharmaceuticals.

The invention is also concerned with novel compounds of structural formula I.

The invention is also concerned with pharmaceutical formulations comprising one of the compounds as an active ingredient.

The invention is further concerned with processes for preparing the compounds of this invention.

### BACKGROUND OF THE INVENTION

Marijuana (Cannabis sativa L.) and its derivatives have been used for centuries for medicinal and recreational purposes. A major active ingredient in marijuana and hashish has been determined to be Δ⁹-tetrahydrocannabinol (Δ⁹-THC). Detailed research has revealed that the biological action of Δ⁹-THC and other members of the cannabinoid family occurs through two G-protein coupled receptors termed CB1 and CB2. The CB1 receptor is primarily found in the central and peripheral nervous systems and to a lesser extent in several peripheral organs. The CB2 receptor is found primarily in lymphoid tissues and cells. Three endogenous ligands for the cannabinoid receptors derived from arachidonic acid have been identified (anandamide, 2-arachidonoyl glycerol, and 2-arachidonyl glycerol ether). Each is an agonist with activities similar to Δ⁹-THC, including sedation, hypothermia, intestinal immobility, antinociception, analgesia, catalepsy, anti-emesis, and appetite stimulation.

The genes for the respective cannabinoid receptors have each been disrupted in mice. The CB1−/− receptor knockout mice appeared normal and fertile. They were resistant to the effects of Δ⁹-THC and demonstrated a strong reduction in the reinforcing properties of morphine and the severity of withdrawal syndrome. They also demonstrated reduced motor activity and hypoalgesia. The CB2−/− receptor knockout mice were also healthy and fertile. They were not resistant to the central nervous system mediated effects of administered Δ⁹-THC. There were some effects on immune cell activation, reinforcing the role for the CB2 receptor in immune system functions.

Excessive exposure to Δ⁹-THC can lead to overeating, psychosis, hypothermia, memory loss, and sedation. Specific synthetic ligands for the cannabinoid receptors have been developed and have aided in the characterization of the cannabinoid receptors: CP55,940 (J. Pharrnacol. Exp. Ther. 1988, 247, 1046-1051); WIN55212-2 (J. Pharmacol. Exp. Ther. 1993, 264, 1352-1363); SR141716A (FEBS Lett. 1994, 350, 240-244; Life Sci. 1995, 56, 1941-1947); and SR144528 (J. Pharmacol. Exp. Ther. 1999, 288, 582-589). The pharmacology and therapeutic potential for cannabinoid receptor ligands has been reviewed (Exp. Opin. Ther. Patents 1998, 8, 301-313; Ann. Rep. Med. Chem., A. Doherty, Ed.; Academic Press, NY 1999, Vol. 34, 199-208; Exp. Opin. Ther. Patents 2000, 10, 1529-1538; Trends in Pharma. Sci. 2000, 21, 218-224). There is at least one CB1 modulator characterized as an inverse agonist or an antagonist, N-(1-piperidinyl)-5-(4-chlorophenyl)-1-(2,4-dichlorophenyl)-4-methylpyrazole-3-carboxamide (SR141716A), in clinical trials for treatment of eating disorders at this time. There still remains a need for potent low molecular weight CB1 modulators that have pharmacokinetic and pharmacodynamic properties suitable for use as human pharmaceuticals.

US Patents U.S. Pat. No. 5,624,941 and U.S. Pat. No. 6,028,084, PCT Application Nos. WO 98/43636 and WO 98/43635, and EPO Application No. EP-658546 disclose substituted pyrazoles having activity against the cannabinoid receptors.

PCT Application Nos. WO 98/31227, WO 98/41519 and WO 02/076949 also disclose substituted pyrazoles having activity against the cannabinoid receptors.

PCT Application Nos. WO 98/37061, WO 00/10967, and WO 00/10968 disclose diaryl ether sulfonamides having activity against the cannabinoid receptors.

PCT Application Nos. WO 97/29079 and WO 99/02499 disclose alkoxy-isoindolones and alkoxy-quinolones as having activity against the cannabinoid receptors.

US patent U.S. Pat. No. 5,532,237 discloses N-benzoyl-indole derivatives having activity against the cannabinoid receptors.

US patents U.S. Pat. No. 4,973,587, U.S. Pat. No. 5,013,837, U.S. Pat. No. 5,081,122, and U.S. Pat. No. 5,112,820, U.S. Pat. No. 5,292,736 disclose aminoalkylindole derivatives as having activity against the cannabinoid receptors.

Treatment of asthma with CB-1 receptor modulators (such as CB-1 inverse agonists) is supported by the finding that presynaptic cannabinoid CB1 receptors mediate the inhibition of noradrenaline release (in the guinea pig lung) (Europ. J. of Pharmacology, 2001, 431 (2), 237-244).

Treatment of cirrhosis of the liver with CB-1 receptor modulators is supported by the finding that a CB1 receptor modulator will reverse the low blood pressure observed in rats with carbon tetrachloride-induced liver cirrhosis and will lower the elevated mesenteric blood flow and portal vein pressure (Nature Medicine, 2001, 7 (7), 827-832).

The compounds of the present invention are modulators of the Cannabinoid-1 (CB1) receptor and are useful in the treatment, prevention and suppression of diseases mediated by the Cannabinoid-1 (CB1) receptor. In particular, compounds of the present invention are antagonists or inverse agonists of the CB1 receptor. The invention is concerned with the use of these compounds to modulate the Cannabinoid-1 (CB1) receptor. As such, compounds of the present invention are useful as psychotropic drugs in the treatment of psychosis, memory deficits, cognitive disorders, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, particularly to opiates, alcohol, marijuana, and nicotine. The compounds are also useful for the treatment of eating disorders by inhibiting excessive food intake and the resulting obesity and complications associated therewith. The compounds are also useful for the treatment of constipation and chronic intestinal pseudo-obstruction, as well as for the treatment of asthma, and cirrhosis of the liver.

### DETAILED DESCRIPTION OF THE INVENTION

The compounds used in the methods of the present invention are represented by the compound of structural formula I:

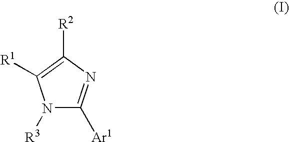

or a pharmaceutically acceptable salt thereof, wherein:

R¹ is selected from:

(1) hydrogen,

(2) C₁₋₄alkyl,

(3) C₂₋₄alkenyl,

(4) C₂₋₄alkynyl,

(5) C₃₋₇cycloalkyl,

(6) C₃₋₇cycloalkyl-C₁₋₄alkyl,

(7) cycloheteroalkyl,

(8) cycloheteroalkyl-C₁₋₄alkyl,

(9) aryl, and

(10) heteroaryl,

wherein alkyl, alkenyl, alkynyl, cycloalkyl, and cycloheteroalkyl are optionally substituted with one to four substituents independently selected from R^{a}, and aryl and heteroaryl are optionally substituted with one to four substituents independently selected from R^{b};

R² is selected from:

(1) —OR^{c},

(2) —OC(O)R^{c},

(3) —OC(O)NR^{c}R^{d},

(4) —SR^{c},

(5) —S(O)ₘR^{c},

(6) —SO₂NR^{c}R^{d},

(7) —NR^{c}R^{d},

(8) —NR^{c}C(O)R^{d},

(9) —NR^{c}SO₂R^{d},

(10) —NR^{c}C(O)NR^{c}R^{d},

(11) —NR^{c}C(O)OR^{d},

(12) —C(O)OR^{c}, and

(13) —C(O)NR^{c}R^{d};

R³ is selected from:

(1) —C₁₋₁₀alkyl, and

(2) —Ar²;

Ar¹ and Ar² are independently selected from phenyl, naphthyl, thienyl, furanyl, pyrrolyl, benzothienyl, benzofuranyl, indanyl, indenyl, indolyl, tetrahydronaphthyl, 2,3-dihydrobenzofuranyl, dihydrobenzopyranyl, and 1,4-benzodioxanyl, each optionally substituted with one, two, or three groups independently selected from R^{b};

Each R^{a} is independently selected from:

(1) —OR^{c},

(2) —NR^{c}S(O)ₘR^{d},

(3) halogen,

(4) —S(O)ₘR^{c},

(5) —SR^{c},

(6) —S(O)₂OR^{c},

(7) —S(O)ₘNR^{c}R^{d},

(8) —NR^{c}R^{d},

(9) —O(CR^{e}R^{f})ₙNR^{c}R^{d},

(10) —C(O)R^{c},

(11) —CO₂R^{c},

(12) —CO₂(CR^{e}R^{f})ₙCONR^{c}R^{d},

(13) —OC(O)R^{c},

(14) —CN,

(15) —C(O)NR^{c}R^{d},

(16) —NR^{c}C(O)R^{d},

(17) —OC(O)NR^{c}R^{d},

(18) —NR^{c}C(O)OR^{d},

(19) —NR^{c}C(O)NR^{c}R^{d},

(20) —CR^{c}(N—OR^{d}),

(21) —CF₃,

(22) —OCF₃,

(23) C₃₋₈cycloalkyl, and

(24) cycloheteroalkyl;

Each R^{b} is independently selected from:

(1) C₁₋₁₆alkyl,

(2) C₂₋₆alkenyl,

(3) C₂₋₆alkynyl,

(4) —OR^{c},

(5) —NR^{c}S(O)ₘR^{d},

(6) —NO₂,

(7) halogen,

(8) —S(O)ₘR^{c},

(9) —SR^{c},

(10) —S(O)₂OR^{c},

(11) —S(O)ₘNR^{c}R^{d},

(12) —NR^{c}R^{d},

(13) —O(CR^{e}R^{f})ₙNR^{c}R^{d},

(14) —C(O)R^{c},

(15) —CO₂R^{c},

(16) —CO₂(CR^{e}R^{f})ₙCONR^{c}R^{d},

(17) —OC(O)R^{c},

(18) —CN,

(19) —C(O)NR^{c}R^{d},

(20) —NR^{c}C(O)R^{d},

(21) —OC(O)NR^{c}R^{d},

(22) —NR^{c}C(O)OR^{d},

(23) —NR^{c}C(O)NR^{c}R^{d},

(24) —CR^{c}(N—OR^{d}),

(25) —CF₃,

(26) —OCF₃,

(27) C₃₋₈cycloalkyl,

(28) cycloheteroalkyl, and

(29) phenyl;

R^{c} and R^{d} are independently selected from:

(1) hydrogen,

(2) C₁₋₁₀alkyl,

(3) C₂₋₁₀alkenyl,

(4) C₂₋₁₀alkynyl,

(5) —NH(C₁₋₁₀alkyl),

(6) —N(C₁₋₁₀alkyl)₂,

(7) cycloalkyl,

(8) cycloalkyl-C₁₋₁₀alkyl;

(9) cycloheteroalkyl,

(10) cycloheteroalkyl-C₁₋₁₀alkyl;

(11) aryl,

(12) heteroaryl,

(13) aryl-C₁₋₁₀alkyl, and

(14) heteroaryl-C₁₋₁₀alkyl, or

R^{c} and R^{d} together with the nitrogen atom to which they are attached form a heterocyclic ring of 4 to 7 members containing 0-2 additional heteroatoms independently selected from oxygen, sulfur and N—R^{c},

each R^{c} and R^{d} may be unsubstituted or substituted with one to three substituents selected from R^{e};

R^{e} and R^{f} are independently selected from:

(1) hydrogen,

(2) C₁₋₁₀alkyl,

(3) C₂₋₁₀alkenyl,

(4) C₂₋₁₀alkynyl,

(5) cycloalkyl,

(6) cycloalkyl-C₁₋₁₀alkyl,

(7) cycloheteroalkyl,

(8) cycloheteroalkyl-C₁₋₁₀alkyl,

(9) aryl,

(10) heteroaryl,

(11) aryl-C₁₋₁₀alkyl, and

(12) heteroaryl-C₁₋₁₀alkyl, or

R^{e} and R^{f} together with the carbon to which they are attached form a ring of 5 to 7 members containing 0-2 heteroatoms independently selected from oxygen, sulfur and nitrogen;

m is selected from 1 and 2; and

n is selected from 1, 2, and 3.

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic or bridged saturated carbocyclic rings, each of which having from 3 to 10 carbon atoms. The term also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. The term also includes aryl group fused to a monocyclic cycloalkyl or monocyclic cycloheteroalkyl group in which the point of attachment is on the aromatic portion. Examples of aryl include phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” means a mono- or bicyclic aromatic ring containing at least one heteroatom selected from N, O and S, with each ring containing 5 to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like. The heteroaryl ring may be substituted on one or more carbon atoms.

“Cycloheteroalkyl” means mono- or bicyclic or bridged saturated rings containing at least one heteroatom selected from N, S and O, each of said ring having from 3 to 10 atoms in which the point of attachment may be carbon or nitrogen. The term also includes monocyclic heterocycle fused to an aryl or heteroaryl group in which the point of attachment is on the non-aromatic portion. Examples of “cycloheteroalkyl” include pyrrolidinyl, piperidinyl, piperazinyl, imidazolidinyl, 2,3-dihydrofuro(2,3-b)pyridyl, benzoxazinyl, tetrahydrohydroquinolinyl, morpholinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The cycloheteroalkyl ring may be substituted on the ring carbons and/or the ring nitrogens.

“Halogen” includes fluorine, chlorine, bromine and iodine.

When any variable (e.g., R¹, R^{d}, etc.) occurs more than one time in any constituent or in formula I, its definition on each occurrence is independent of its definition at every other occurrence. Also, combinations of substituents and/or variables are permissible only if such combinations result in stable compounds.

Under standard nomenclature used throughout this disclosure, the terminal portion of the designated side chain is described first, followed by the adjacent functionality toward the point of attachment. For example, a C₁₋₅ alkylcarbonylamino C₁₋₆alkyl substituent is equivalent to

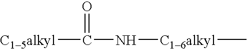

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Compounds of Formula I may contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula I. By way of illustration, tautomers included in this definition include, but are not limited to:

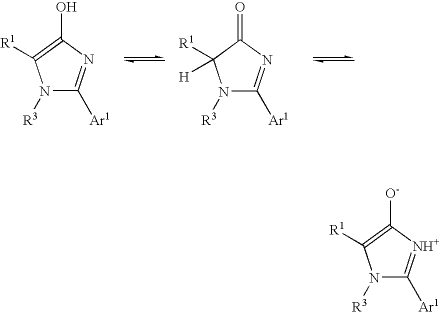

Compounds of the Formula I may be separated into diastereoisomeric pairs of enantiomers by, for example, fractional crystallization from a suitable solvent, for example MeOH or ethyl acetate or a mixture thereof. The pair of enantiomers thus obtained may be separated into individual stereoisomers by conventional means, for example by the use of an optically active amine as a resolving agent or on a chiral HPLC column.

Alternatively, any enantiomer of a compound of the general Formula I may be obtained by stereospecific synthesis using optically pure starting materials or reagents of known configuration.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamnine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to the compounds of Formula I are meant to also include the pharmaceutically acceptable salts.

In one embodiment of the present invention, R¹ is selected from:

(1) hydrogen,

(2) C₁₋₄alkyl,

(3) C₂₋₄alkenyl,

(4) C₂₋₄alkynyl,

(5) C₃₋₇cycloalkyl, and

(6) C₃₋₇cycloalkyl-C₁₋₄alkyl;

wherein alkyl, alkenyl, alkynyl, and cycloalkyl, are optionally substituted with one to four substituents independently selected from R^{a}.

In one class of this embodiment of the present invention, R¹ is selected from:

(1) hydrogen, and

(2) C₁₋₄alkyl;

wherein alkyl is optionally substituted with one to four substituents independently selected from R^{a}.

In a subclass of this class of the present invention, R¹ is selected from:

(1) hydrogen,

(2) methyl, and

(3) ethyl;

wherein methyl and ethyl are optionally substituted with one to four substituents independently selected from R^{a}.

In another subclass of this class of the present invention, R¹ is selected from:

(1) methyl, and

(2) ethyl;

wherein methyl and ethyl are optionally substituted with one to four substituents independently selected from R^{a}.

In still another subclass of the present invention, R¹ is methyl, wherein methyl is optionally substituted with one to three substituents independently selected from R^{a}.

In another embodiment of the present invention, R² is selected from:

(1) —OR^{c},

(2) —SR^{c},

(3) —S(O)ₘR^{c},

(4) —SO₂NR^{c}R^{d},

(5) —NR^{c}R^{d},

(6) —NR^{c}C(O)R^{d},

(7) —NR^{c}SO₂R^{d},

(8) —C(O)OR^{c}, and

(9) —C(O)NR^{c}R^{d}.

In one class of this embodiment of the present invention, R² is selected from:

(1) —OR^{c},

(2) —NR^{c}R^{d},

(3) —NR^{c}C(O)R^{d},

(4) —NR^{c}SO₂R^{d},

(5) —C(O)OR^{c}, and

(6) —C(O)NR^{c}R^{d}.

In one subclass of this class of the present invention, R² is

(1) —OR^{c},

(2) —NR^{c}C(O)R^{d},

(3) —C(O)OR^{c}, and

(4) —C(O)NR^{c}R^{d}.

In another subclass of this class of the present invention, R² is selected from —C(O)NR^{c}R^{d}.

In another embodiment of the present invention, R³ is selected from:

(1) —C₁₋₄alkyl, and

(2) —Ar².

In one class of this embodiment of the present invention, R³ is selected from:

(1) methyl, and

(2) —Ar².

In a subclass of this class of the present invention, R³ is Ar².

In another embodiment of the present invention, Ar¹ and Ar² are independently selected from phenyl, naphthyl, thienyl, each optionally substituted with one or two groups independently selected from R^{b}.

In one class of this embodiment of the present invention, Ar¹ and Ar² are phenyl, each optionally substituted with one or two groups independently selected from R^{b}.

In a subclass of this class of the present invention, Ar¹ and Ar² are each independently selected from:

(1) phenyl,

(2) 4-fluorophenyl,

(3) 2-chlorophenyl,

(4) 3-chlorophenyl,

(5) 4-chlorophenyl,

(6) 4-cyanophenyl,

(7) 4-methylphenyl,

(8) 4-isopropylphenyl,

(9) 4-biphenyl,

(10) 4-bromophenyl,

(11) 4-iodophenyl,

(12) 2,4-dichlorophenyl, and

(13) 2-chloro-4-fluorophenyl.

In another subclass of this class of the present invention, Ar¹ is 2,4-dichlorophenyl, and Ar² is 4-chlorophenyl.

In another embodiment of the present invention, each R^{a} is independently selected from:

(1) —OR^{c},

(2) —NR^{c}S(O)ₘR^{d},

(3) halogen,

(4) —S(O)ₘR^{c},

(5) —SR^{c},

(6) —S(O)ₘNR^{c}R^{d},

(7) —NR^{c}R^{d},

(8) —O(CR^{e}R^{f})ₙNR^{c}R^{d},

(9) —C(O)R^{c},

(10) —CN,

(11) —C(O)NR^{c}R^{d},

(12) —NR^{c}C(O)R^{d},

(13) —CF₃,

(14) —OCF₃,

(15) —C₃₋₈cycloalkyl, and

(16) cycloheteroalkyl.

In another embodiment of the present invention, each R^{b} is independently selected from:

(1) C₁₋₆alkyl,

(2) —OR^{c},

(3) halogen,

(4) —CN,

(5) —C(O)NR^{c}R^{d},

(6) —NR^{c}C(O)R^{d},

(7) —CF₃,

(8) —OCF₃, and

(9) phenyl

In one class of this embodiment of the present invention, each R^{b} is independently selected from:

(1) halogen,

(2) C₁₋₃alkyl,

(3) —CN, and

(4) phenyl.

In another embodiment of the present invention, at each occurrence R^{c} and R^{d} are independently selected from:

(1) hydrogen,

(2) —C₁₋₁₀alkyl,

(3) —NH(C₁₋₁₀alkyl),

(4) —N(C₁₋₁₀alkyl)₂,

(5) cycloalkyl, and

(6) cycloheteroalkyl, or

R^{c} and R^{d} together with the nitrogen atom to which they are attached form a heterocyclic ring of 4 to 7 members containing 0-2 additional heteroatoms independently selected from oxygen, sulfur and N—R^{c}, each R^{c} and R^{d} may be unsubstituted or substituted with one to three substituents selected from R^{e}.

In one class of this embodiment of the present invention, at each occurrence R^{c} and R^{d} are independently selected from:

(1) hydrogen,

(2) methyl,

(3) ethyl,

(4) —N(CH₃)₂,

(5) —NH(CH₃),

(6) cyclopentane,

(7) cyclohexane,

(8) cycloheptane,

(9) piperidine

(10) morpholine,

(11) pyrrolidine,

(12) azepine, and

(13) 4-methylpiperazine,

each R^{c} and R^{d} may be unsubstituted or substituted with one to three substituents selected from R^{e}.

In a subclass of this class of the present invention, at each occurrence R^{c} and R^{d} are independently selected from:

(1) hydrogen,

(2) cyclohexane, and

(3) piperidine,

each R^{c} and R^{d} may be unsubstituted or substituted with one to three substituents selected from R^{e}.

In another embodiment of the present invention, at each occurrence R^{e} and R^{f} are independently selected from:

(1) hydrogen,

(2) C₁₋₁₀alkyl,

(3) cycloheteroalkyl,

(4) cycloheteroalkyl-C₁₋₁₀alkyl,

(5) aryl,

(6) heleroaryl,

(7) aryl-C₁₋₁₀alkyl, and

(8) heteroaryl-C₁₋₁₀alkyl, or

R^{e} and R^{f} together with the carbon to which they are attached form a ring of 5 to 7 members containing 0-2 heteroatoms independently selected from oxygen, sulfur and nitrogen.

Particular novel compounds which may be employed in the methods, uses and compositions of the present invention, include:

(1) ethyl 1-(4-chlorophenyl)-2-(2,4-dichlorophenyl)-5-methyl-imidazole-4-carboxylate;

(2) ethyl 2-(2,4-dichlorophenyl)-1,5-dimethyl-imidazole-4-carboxylate;

(3) N-(cyclohexyl)-1-(4-chlorophenyl)-2-(2,4-dichlorophenyl)-5-methyl-imidazole-4-carboxamide;

(4) N-(piperidin-1-yl)-1-(4-chlorophenyl)-2-(2,4-dichlorophenyl)-5-methyl-imidazole-4-carboxamide;

(5) N-(piperidin-1-yl)-2-(2,4-dichlorophenyl)-1,5-dimethyl-imidazole-4-carboxamide;

(6) N-(cyclopentyl)-1-(4-chlorophenyl)-2-(2,4-dichlorophenyl)-5-methyl-imidazole-4-carboxamide;

(7) N-(cycloheptyl)-1-(4-chlorophenyl)-2-(2,4-dichlorophenyl)-5-methyl-imidazole-4-carboxamide;

(8) N-(morpholin-4-yl)-1-(4-chlorophenyl)-2-(2,4-dichlorophenyl)-5-methyl-i midazole-4-carboxamide;

(9) N-(pyrrolidin-1-yl)-1-(4-chlorophenyl)-2-(2,4-dichlorophenyl)-5-methyl-imidazole-4-carboxamide;

(10) N-(azepin-1-yl)-1-(4-chlorophenyl)-2-(2,4-dichlorophenyl)-5-methyl-imidazole-4-carboxamide;

(11) N-(4-methylpiperazin-1-yl)-1-(4-chlorophenyl)-2-(2,4-dichlorophenyl)-5-methyl-imidazole-4-carboxamide;

(12) N′,N′-dimethyl-1-(4-chlorophenyl)-2-(2,4-dichlorophenyl)-5-methyl-imidazole-4-carboxhydrazide;

(13) N-(cyclohexyl)-1-(phenyl)-2-(2,4-dichlorophenyl)-5-methyl-imidazole-4-carboxamide;

(14) N-(piperidin-1-yl)-1-(phenyl)-2-(2,4-dichlorophenyl)-5-methyl-imidazole-4-carboxamide;

(15) N-(cyclohexyl)-1-(4-fluorophenyl)-2-(2,4-dichlorophenyl)-5-methyl-imidazole-4-carboxamide;

(16) N-(piperidin-1-yl)-1-(4-fluorophenyl)-2-(2,4-dichlorophenyl)-5-methyl-imidazole-4-carboxamide;

(17) N-(4-methyl-cyclohexyl)-1-(4-chlorophenyl)-2-(2,4-dichlorophenyl)-5-methyl-imidazole-4-carboxamide (Isomer A);

(18) N-(2-(pyrrolidin-1-yl)ethyl)-1-(4-chlorophenyl)-2-(2,4-dichlorophenyl)-5-methyl-imidazole-4-carboxamide;

(19) N-(cyclohexyl)-1-(4-chlorophenyl)-2-(2-chlorophenyl)-5-methyl-imidazole-4-carboxamide;

(20) N-(piperidin-1-yl)-1-(4-chlorophenyl)-2-(2-chlorophenyl)-5-methyl-imidazole-4-carboxamide;

(21) N-(cyclohexyl)-1-(4-bromophenyl)-2-(2,4-dichlorophenyl)-5-methyl-imidazole-4-carboxamide;

(22) N-(piperidin-1-yl)-1-(4-bromophenyl)-2-(2,4-dichlorophenyl)-5-methyl-imidazole-4-carboxamide;

(23) N-(cyclohexyl)-1-(4-isopropylphenyl)-2-(2,4-dichlorophenyl)-5-methyl-imidazole-4-carboxamide;

(24) N-(piperidin-1-yl)-1-(4-isopropylphenyl)-2-(2,4-dichlorophenyl)-5-methyl-imidazole-4-carboxamide;

(25) N-(cyclohexyl)-1-(4-cyanophenyl)-2-(2,4-dichlorophenyl)-5-methyl-imidazole-4-carboxamide;

(26) N-(piperidin-1-yl)-1-(4-cyanophenyl)-2-(2,4-dichlorophenyl)-5-methyl-imidazole-4-carboxamide;

(27) N-(cyclohexyl)-1-(4-biphenyl)-2-(2,4-dichlorophenyl)-5-methyl-imidazole-4-carboxamide;

(28) N-(piperidin-1-yl)-1-(4-biphenyl)-2-(2,4-dichlorophenyl)-5-methyl-imidazole-4-carboxamide;

(29) N-(cyclohexyl)-1,2-bis(4-chlorophenyl)-5-methyl-imidazole-4-carboxamide;

(30) N-(piperidin-1-yl)-1,2-bis(4-chlorophenyl)-5-methyl-imidazole-4-carboxamide;

(31) N-(cyclohexyl)-1-(4-chlorophenyl)-2-(2,4-dichlorophenyl)-5-ethyl-imidazole-4-carboxamide;

(32) N-(piperidin-1-yl)-1-(4-chlorophenyl)-2-(2,4-dichlorophenyl)-5-ethyl-imidazole-4-carboxamide;

(33) N-(cyclohex yl)-1-(4-bromophenyl)-2-(2,4-dichlorophenyl)-5-ethyl-imidazole-4-carboxamide;

(34) N-(piperidin-1-yl)-1-(4-bromophenyl)-2-(2,4-dichlorophenyl)-5-ethyl-imidazole-4-carboxamide;

(35) N-(cyclohexyl)-1-(4-isopropylphenyl)-2-(2,4-dichlorophenyl)-5-ethyl-imidazole-4-carboxamide;

(36) N-(piperidin-1-yl)-1-(4-isopropylphenyl)-2-(2,4-dichlorophenyl)-5-ethyl-imidazole-4-carboxamide;

(37) N-(cyclohexyl)-1-(3-chlorophenyl)-2-(2,4-dichlorophenyl)-5-methyl-imidazole-4-carboxamide;

(38) N-(cyclohexyl)-1-(4-chlorophenyl)-2-(2-chloro-4-fluorophenyl)-5-methyl-imidazole-4-carboxamide;

(39) N-(piperidin-1-yl)-1-(4-chlorophenyl)-2-(2-chloro-4-fluorophenyl)-5-methyl-imidazole-4-carboxamide;

(40) N-(cyclohexyl)-1-(2-chlorophenyl)-2-(2,4-dichlorophenyl)-5-methyl-imidazole-4-carboxamide; and

(41) N-(cyclohexyl)-1-(4-chlorophenyl)-2-(3-chlorophenyl)-5-methyl-imidazole-4-carboxamide;

and pharmaceutically acceptable salts thereof.

In one embodiment of the present invention, a compound selected from the following novel compounds is employed:

(1) N-(cyclohexyl)-1-(4-chlorophenyl)-2-(2,4-dichlorophenyl)-5-methyl-imidazole-4-carboxamide;

(2) N-(piperidin-1-yl)-1-(4-chlorophenyl)-2-(2,4-dichlorophenyl)-5-methyl-imidazole-4-carboxamide;

(3) N-(cyclohexyl)-1-(4-bromophenyl)-2-(2,4-dichlorophenyl)-5-ethyl-imidazole-4-carboxamide;

(4) N-(cyclohexyl)-1-(4-isopropylphenyl)-2-(2,4-dichlorophenyl)-5-ethyl-imidazole-4-carboxamide;

(5) N-(cyclohexyl)-1-(4-bromophenyl)-2-(2,4-dichlorophenyl)-5-methyl-imidazole-4-carboxamide; and

(6) N-(cycloheptyl)-1-(4-chlorophenyl)-2-(2,4-dichlorophenyl)-5-methyl-imidazole-4-carboxamide;

and pharmaceutically acceptable salts thereof.

Compounds of this invention are modulators of the CB1 receptor and as such are useful as psychotropic drugs in the treatment of psychosis, memory deficits, cognitive disorders, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, particularly to opiates, alcohol, marijuana, and nicotine. The compounds are also useful for the treatment of obesity or eating disorders associated with excessive food intake and complications associated therewith. The compounds are also useful for the treatment of constipation and chronic intestinal pseudo-obstruction. The compounds are also useful for the treatment of cirrhosis of the liver. The compounds are also useful for the treatment of asthma.

The terms “administration of” and or “administering a” compound should be understood to mean providing a compound of the invention or a prodrug of a compound of the invention to the individual in need of treatment.

The administration of the compound of structural formula I in order to practice the present methods of therapy is carried out by administering an effective amount of the compound of structural formula I to the patient in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The utilities of the present compounds in these diseases or disorders may be demonstrated in animal disease models that have been reported in the literature. The following are examples of such animal disease models: a) suppression of food intake and resultant weight loss in rats (Life Sciences 1998, 63, 113-117); b) reduction of sweet food intake in marmosets (Behavioural Pharm. 1998, 9, 179-181); c) reduction of sucrose and ethanol intake in mice (Psychopharm. 1997, 132, 104-106); d) increased motor activity and place conditioning in rats (Psychopharm. 1998, 135, 324-332; Psychopharmacol 2000, 151: 25-30); e) spontaneous locomotor activity in mice (J. Pharm. Exp. Ther. 1996, 277, 586-594); f) reduction in opiate self-administration in mice (Sci. 1999, 283,401-404); g) bronchial hyperresponsiveness in sheep and guinea pigs as models for the various phases of asthma (for example, see W. M. Abraham et al., “α₄-Integrins mediate antigen-induced late bronchial responses and prolonged airway hyperresponsiveness in sheep.” J. Clin. Invest. 93, 776 (1993) and A. A. Y. Milne and P. P. Piper, “Role of VLA-4 integrin in leucocyte recruitment and bronchial hyperresponsiveness in the gunea-pig.” Eur. J. Pharmacol., 282, 243 (1995)); h) mediation of the vasodilated state in advanced liver cirrhosis induced by carbon tetrachloride (Nature Medicine, 2001, 7 (7), 827-832); i) amitriptyline-induced constipation in cynomolgus monkeys is beneficial for the evaluation of laxatives (Biol. Pharm. Bulletin (Japan), 2000, 23(5), 657-9); j) neuropathology of paediatric chronic intestinal pseudo-obstruction and animal models related to the neuropathology of paediatric chronic intestinal pseudo-obstruction (Journal of Pathology (England), 2001, 194 (3), 277-88).

The magnitude of prophylactic or therapeutic dose of a compound of Formula I will, of course, vary with the nature of the severity of the condition to be treated and with the particular compound of Formula I and its route of administration. It will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.001 mg to about 100 mg (preferably from 0.01 mg to about 50 mg, more preferably 0.1 mg to 10 mg) of a compound of Formula I per kg of body weight per day.

In the case where an oral composition is employed, a suitable dosage range is, e.g. from about 0.01 mg to about 1000 mg of a compound of Formula I per day, preferably from about 0.1 mg to about 10 mg per day. For oral administration, the compositions are preferably provided in the form of tablets containing from 0.01 to 1,000 mg, preferably 0.01, 0.05, 0.1, 0.5, 1, 2.5, 5, 10, 15, 20, 25, 30, 40, 50, 100, 250, 500, 750 or 1000 milligrams of the active ingredient for the symptomatic adjustment of the dosage to the patient to be treated.

For the treatment of diseases of the eye, ophthalmic preparations for ocular administration comprising 0.001-1% by weight solutions or suspensions of the compounds of Formula I in an acceptable ophthalmic formulation may be used.

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human, with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from 0.01 to 1,000 mg, particularly 0.01, 0.05, 0.1, 0.5, 1, 2.5, 3, 5, 6, 10, 15, 25, 50, 75, 100, 125, 150, 175, 180, 200, 225, 500, 750 and 1,000 milligrams of the active ingredient for the symptomatic adjustment of the dosage to the patient to be treated. and each cachet or capsule contains from about 0.01 to 1,000 mg, particularly 0.01, 0.05, 0.1, 0.5, 1.0, 2.5, 3, 5, 6, 10, 15, 25, 50, 75, 100, 125, 150, 175, 180, 200, 225, 500, 750 and 1,000 milligrams of the active ingredient for the symptomatic adjustment of the dosage to the patient to be treated.

Exemplifying the invention is a pharmaceutical composition comprising any of the compounds described above and a pharmaceutically acceptable carrier. Also exemplifying the invention is a pharmaceutical composition made by combining any of the compounds described above and a pharmaceutically acceptable carrier. An illustration of the invention is a process for making a pharmaceutical composition comprising combining any of the compounds described above and a pharmaceutically acceptable carrier.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdernal routes, by rectal or vaginal suppositories, or through a continual intravenous solution, the dosage administration will, of course, be continuous rather than intermittent throughout the dosage regimen.

The following are examples of representative pharmaceutical dosage forms for the compounds of Formula I:

| Injectable Suspension (I.M.) | mg/mL |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |

Water for injection to a total volume of 1 mL

| Tablet | mg/tablet |
|---|---|
| Compound of Formula I | 25 |
| Microcrystalline Cellulose | 415 |
| Povidone | 14.0 |
| Pregelatinized Starch | 43.5 |
| Magnesium Stearate | 2.5 |
| | 500 |

| Capsule | mg/capsule |
|---|---|
| Compound of Formula 1 | 25 |
| Lactose Powder | 573.5 |
| Magnesium Stearate | 1.5 |
| | 600 |

| Aerosol | Per canister |
|---|---|
| Compound of Formula I | 24 mg |
| Lecithin, NF Liq. Conc. | 1.2 mg |
| Trichlorofluoromethane, NF | 4.025 g |
| Dichlorodifluoromethane, NF | 12.15 g |

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I include, but are not limited to: antipsychotic agents, cognition enhancing agents, anti-migraine agents, anti-asthmatic agents, antiinflammatory agents, axiolytics, anti-Parkinson's agents, anti-epileptics, anorectic agents, serotonin reuptake inhibitors, and other antiobesity agents which may be administered separately or in the same pharmaceutical compositions.

The present invention also provides a method for the treatment or prevention of a CB-1 receptor modulator mediated disease, which method comprises administration to a patient in need of such treatment or at risk of developing a CB-1 receptor modulator mediated disease of an amount of a CB1 receptor modulator and an amount of one or more active ingredients, such that together they give effective relief.

In a further aspect of the present invention, there is provided a pharmaceutical composition comprising a CB1 receptor modulator and one or more active ingredients, together with at least one pharmaceutically acceptable carrier or excipient.

Thus, according to a further aspect of the present invention there is provided the use of a CB1 receptor modulator and one or more active ingredients for the manufacture of a medicament for the treatment or prevention of a CB-1 receptor modulator mediated disease. In a further or alternative aspect of the present invention, there is therefore provided a product comprising a CB1 receptor modulator and one or more active ingredients as a combined preparation for simultaneous, separate or sequential use in the treatment or prevention of CB-1 receptor modulator mediated disease. Such a combined preparation may be, for example, in the form of a twin pack.

It will be appreciated that for the treatment or prevention of eating disorders, including obesity, bulimia nervosa and compulsive eating disorders, a compound of the present invention may be used in conjunction with other anorectic agents.

The present invention also provides a method for the treatment or prevention of eating disorders, which method comprises administration to a patient in need of such treatment an amount of a compound of the present invention and an amount of an anorectic agent, such that together they give effective relief.

Suitable anoretic agents of use in combination with a compound of the present invention include, but are not limited to, aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; and pharmaceutically acceptable salts thereof.

A particularly suitable class of anorectic agent are the halogenated amphetamine derivatives, including chlorphentermine, cloforex, clortermine, dexfenfluramine, fenfluramine, picilorex and sibutramine; and pharmaceutically acceptable salts thereof Particularly preferred halogenated amphetamine derivatives of use in combination with a compound of the present invention include: fenfluramine and dexfenfluramine, and pharmaceutically acceptable salts thereof.

It will be appreciated that for the treatment or prevention of obesity, the compounds of the present invention may also be used in combination with a selective serotonin reuptake inhibitor (SSRI).

The present invention also provides a method for the treatment or prevention of obesity, which method comprises administration to a patient in need of such treatment an amount of a compound of the present invention and an amount of an SSRI, such that together they give effective relief.

Suitable selective serotonin reuptake inhibitors of use in combination with a compound of the present invention include: fluoxetine, fluvoxamine, paroxetine, sertraline, and imipramine and pharmaceutically acceptable salts thereof.

It will be appreciated that for the treatment or prevention of obesity, the compounds of the present invention may also be used in combination with an opioid antagonist.

The present invention also provides a method for the treatment or prevention of obesity, which method comprises administration to a patient in need of such treatment an amount of a compound of the present invention and an amount of an opioid antagonist, such that together they give effective relief.

Suitable opioid antagonists of use in combination with a compound of the present invention include: naltrexone, 3-methoxynaltrexone, naloxone and nalmefene, and pharmaceutically acceptable salts thereof.

It will be appreciated that for the treatment or prevention of obesity, the compounds of the present invention may also be used in combination with another anti-obesity agent.

The present invention also provides a method for the treatment or prevention of obesity, which method comprises administration to a patient in need of such treatment an amount of a compound of the present invention and an amount of another anti-obesity agent, such that together they give effective relief.

Suitable anti-obesity agents of use in combination with a compound of the present invention, include, but are not limited to: 1) growth hormone secretagogues, such as those disclosed and specifically described in U.S. Pat. No. 5,536,716; 2) growth hormone secretagogue receptor agonists/antagonists, such as NN703, hexarelin, MK-0677, SM-130686, CP-424,391, L-692,429 and L-163,255, and such as those disclosed in U.S. Pat. No. 6,358,951, U.S. patent application Ser. Nos. 2002/049196 and 2002/022637, and PCT Application Nos. WO 01/56592 and WO 02/32888; 3) melanocortin agonists, such as Melanotan II or those described in WO 99/64002 and WO 00/74679; 4) Mc4r (melanocortin 4 receptor) agonists, such as CHIR86036 (Chiron), ME-10142, and ME-10145 (Melacure), and those disclosed in PCT Application Nos. WO 01/991752, WO 01/74844, WO 02/12166, WO 02/11715, and WO 02/12178; 5) β-3 agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, SR 59119A, and such as those disclosed in U.S. patent application Ser. Nos. 5,705,515, and U.S. Pat. No. 5,451,677 and PCT Patent Publications WO 94/18161, WO 95/29159, WO 97/46556, WO 98/04526 and WO 98/32753, WO 01/74782, and WO 02/32897; 6) 5HT-2 agonists; 7) 5HT2C (serotonin receptor 2C) agonists, such as BVT933, DPCA37215, WAY161503, R-1065, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; 8) orexin antagonists, such as SB-334867-A, and those disclosed in PCT Patent Application Nos. WO 01/96302, WO 01/68609, WO 02/51232, and WO 02/51838; 9) melanin concentrating hormone antagonists; 10) melanin-concentrating hormone 1 receptor (MCH1R) antagonists, such as T-226296 (Takeda), and those disclosed in PCT Patent Application Nos. WO 01/82925, WO 01/87834, WO 02/06245, WO 02/04433, and WO 02/51809, and Japanese Patent Application No. JP 13226269; 11) melanin-concentrating hormone 2 receptor (MCH2R) agonist/antagonists; 12) galanin antagonists; 13) CCK agonists; 14) CCK-A (cholecystokinin-A) agonists, such as AR-R 15849, GI 181771, JMV-180, A-71378, A-71623 and SR146131, and those discribed in U.S. Pat. No. 5,739,106; 15) GLP-1 agonists; 16) corticotropin-releasing hormone agonists; 17) NPY 5 antagonists, such as GW-569180A, GW-594884A, GW-587081X, GW-548118X, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,140,354, 6,191,160, 6,313,298, 6,337,332, 6,329,395, 6,326,375, 6,335,345, and 6,340,683, European Patent Nos. EP-01010691, and EP-01044970, and PCT Patent Publication Nos. WO 97/19682, WO 97/20820, WO 97/20821, WO 97/20822, WO 97/20823, WO 98/27063, WO 00/64880, WO 00/68197, WO 00/69849, WO 01/09120, WO 01/14376, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; 18) NPY 1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; 19) histamine receptor-3 (H3) modulators; 20) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, GT2394 (Gliatech), and those described and disclosed in PCT Application No. WO 02/15905, and O-[3-(1H-imidazol-4-yl)propanol]-carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm.(Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)); 21) β-hydroxy steroid dehydrogenase-1 inhibitors (β-HSD-1); 22) PDE (phosphodiesterase) inhibitors, such as theophylline, pentoxifylline, zaprinast, sildenafil, amnrinone, milrinone, cilostamide, rolipram, and cilomilast; 23) phosphodiesterase-3B (PDE3B) inhibitors; 24) NE (norepinephrine) transport inhibitors, such as GW 320659, despiramine, talsupram, and nomifensine; 25) non-selective serotonin/norepinephrine transport inhibitors, such as sibutramine or fenfluramine; 26) ghrelin antagonists, such as those disclosed in PCT Application Nos. WO 01/87335, and WO 02/08250; 27) leptin, including recombinant human leptin (PEG-OB, Hoffman La Roche) and recombinant methionyl human leptin (Amgen); 28) leptin derivatives, such as those disclosed in U.S. Pat. Nos. 5,552,524, 5,552,523, 5,552,522, 5,521,283, and PCT International Publication Nos. WO 96/23513, WO 96/23514, WO 96/23515, WO 96/23516, WO 96/23517, WO 96/23518, WO 96/23519, and WO 96/23520; 29) BRS3 (bombesin receptor subtype 3) agonists; 30) CNTF (Ciliary neurotrophic factors), such as GI-181771 (Glaxo-SmithKline), SR146131 (Sanofi Synthelabo), butabindide, PD170,292, and PD 149164 (Pfizer); 31) CNTF derivatives, such as axokine (Regeneron), and those disclosed in PCT Application Nos. WO 94/09134, WO 98/22128, and WO 99/43813; 32) monoamine reuptake inhibitors, such as those disclosed in PCT Application Nos. WO 01/27068, and WO 01/62341; 33) UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in PCT Patent Application No. WO 99/00123; 34) thyroid hormone β agonists, such as KB-2611 (KaroBioBMS), and those disclosed in PCT Application No. WO 02/15845, and Japanese Patent Application No. JP 2000256190; 35) FAS (fatty acid synthase) inhibitors, such as Cerulenin and C75; 36) DGAT1 (diacylglycerol acyltransferase 1) inhibitors; 37) DGAT2 (diacylglycerol acyltransferase 2) inhibitors; 38) ACC2 (acetyl-CoA carboxylase-2) inhibitors; 39) glucocorticoid antagonists; 40) acyl-estrogens, such as oleoyl-estrone, disclosed in del Mar-Grasa, M. et al., Obesity Research, 9:202-9 (2001); 41) lipase inhibitors, such as orlistat (Xenical®), Triton WR1339, RHC80267, lipstatin, tetrahydrolipstatin, teasaponin, diethylumbelliferyl phosphate, and those disclosed in PCT Application No. WO 01/77094; 42) fatty acid transporter inhibitors; 43) dicarboxylate transporter inhibitors; 44) glucose transporter inhibitors; 45) phosphate transporter inhibitors; 46) serotonin reuptake inhibitors, such as those disclosed in U.S. patent application Ser. No. 6,365,633, and PCT Patent Application Nos. WO 01/27060, and WO 01/162341; 47) Metformin (Glucophage@)); and/or 48) Topiramate (Topimax®).

Specific NPY5 antagonists of use in combination with a compound of the present invention are selected from the group consisting of:

(1) 3-oxo-N-(5-phenyl-2-pyrazinyl)-spiro[isobenzofuran-1(3H),4′-piperidine]-1′-carboxamide,

(2) 3-oxo-N-(7-trifluoromethylpyrido[3,2-b]pyridin-2-yl)spiro-[isobenzofuran-1(3H),4′-piperidine]-1′-carboxamide,

(3) N-[5-(3-fluorophenyl)-2-pyrimidinyl]-3-oxospiro-[isobenzofuran-1(3H),4′-piperidine]-1′-carboxamide,

(4) trans-3′-oxo-N-(5-phenyl-2-pyrimidinyl)spiro[cyclohexane-1,1′(3′H)-isobenzofuran]-4-carboxamide,

(5) trans-3′-oxo-N-[1-(3-quinolyl)-4-imidazolyl]spiro[cyclohexane-1,1′(3′H)-isobenzofuran]-4-carboxamide,

(6) trans-3-oxo-N-(5-phenyl-2-pyrazinyl)spiro[4-azaiso-benzofuran-1 (3H),1′-cyclohexane]-4′-carboxamide,

(7) trans-N-[5-(3-fluorophenyl)-2-pyrimidinyl]-3-oxospiro[5-azaisobenzofuran-1(3H), 1′-cyclohexane]-4′-carboxamide,

(8) trans-N-[5-(2-fluorophenyl)-2-pyrimidinyl]-3-oxospiro[5-azaisobenzofuran-1(3H), 1′-cyclohexane]-4′-carboxamide,

(9) trans-N-[1-(3,5-difluorophenyl)-4-imidazolyl]-3-oxospiro[7-azaisobenzofuran-1(3H), 1′-cyclohexane]-4′-carboxamide,

(10) trans-3-oxo-N-(1-phenyl-4-pyrazolyl)spiro[4-azaisobenzofuran-1(3H), 1′-cyclohexane]-4′-carboxamide,

(11) trans-N-[1-(2-fluorophenyl)-3-pyrazolyl]-3-oxospiro[6-azaisobenzofuran-1(3H), 1′-cyclohexane]-4′-carboxamide,

(12) trans-3-oxo-N-(1-phenyl-3-pyrazolyl)spiro[6-azaisobenzofuran-1(3H), 1′-cyclohexane]-4′-carboxamide,

(13) trans-3-oxo-N-(2-phenyl-1,2,3-triazol-4-yl)spiro[6-azaisobenzofuran-1(3H), 1′-cyclohexane]-4′-carboxamide, and pharmaceutically acceptable salts and esters thereof.

As used herein “obesity” refers to a condition whereby a mammal has a Body Mass Index (BMI), which is calculated as weight per height squared (kg/m²), of at least 30. For humans, conventionally, those persons with normal healthy weight have a BMI of 19.9 to less than 25. Overweight persons have a BMI of 25 to less than 30. “A person at risk of obesity” is an overweight person with a BMI of 25 to less than 30.

The obesity herein may be due to any cause, whether genetic or environmental. Examples of disorders that may result in obesity or be the cause of obesity include overeating and bulimia, polycystic ovarian disease, craniopharyngioma, the Prader-Willi Syndrome, Frohlich's syndrome, Type II diabetes, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia.

“Treatment” (of obesity) refers to reducing the BMI of the mammal to less than about 30, and maintaining that weight for at least 6 months. The treatment suitably results in a reduction in food or calorie intake by the mammal.

“Prevention” (of obesity) refers to preventing obesity from occurring if the treatment is administered prior to the onset of the obese condition in overweight persons with a BMI between 25 and 30. Moreover, if treatment is commenced in already obese subjects, such treatment is expected to prevent, or to prevent the progression of, the medical sequelae of obesity, such as, e.g., arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

It will be appreciated that for the treatment or prevention of migraine, a compound of the present invention may be used in conjunction with other anti-migraine agents, such as ergotamines or 5-HT₁ agonists, especially sumatriptan, naratriptan, zolmatriptan or rizatriptan.

It will be appreciated that for the treatment of depression or anxiety, a compound of the present invention may be used in conjunction with other anti-depressant or anti-anxiety agents.

Suitable classes of anti-depressant agents include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRls), corticotropin releasing factor (CRF) antagonists, α-adrenoreceptor antagonists, neurokinin-1 receptor antagonists and atypical anti-depressants.

Suitable norepinephrine reuptake inhibitors include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Suitable selective serotonin reuptake inhibitors include: fluoxetine, fluvoxamine, paroxetine and sertraline, and pharmaceutically acceptable salts thereof.

Suitable monoamine oxidase inhibitors include: isocarboxazid, pheneizine, tranylcypromine and selegiline, and pharmaceutically acceptable salts thereof.

Suitable reversible inhibitors of monoamine oxidase include: moclobemide, and pharmaceutically acceptable salts thereof.

Suitable serotonin and noradrenaline reuptake inhibitors of use in the present invention include: venlafaxine, and pharmaceutically acceptable salts thereof.

Suitable CRF antagonists include those compounds described in International Patent Specification Nos. WO 94/13643, WO 94/13644, WO 94/13661, WO 94/13676 and WO 94/13677.

Suitable neurokinin-1 receptor antagonists may be peptidal or non-peptidal in nature, however, the use of a non-peptidal neurokinin-1 receptor antagonist is preferred. In a preferred embodiment, the neurokinin-1 receptor antagonist is a CNS-penetrant neurokinin-1 receptor antagonist. In addition, for convenience the use of an orally active neurokinin-1 receptor antagonist is preferred. To facilitate dosing, it is also preferred that the neurokinin-1 receptor antagonist is a long acting neurokinin-l receptor antagonist. An especially preferred class of neurokinin-1 receptor antagonists of use in the present invention are those compounds which are orally active and long acting.

Neurokinin-1 receptor antagonists of use in the present invention are fully described, for example, in U.S. Pat. Nos. 5,162,339, 5,232,929, 5,242,930, 5,373,003, 5,387,595, 5,459,270, 5,494,926, 5,496,833, 5,637,699; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, and 97/49710; and in British Patent Publication Nos. 2 266 529, 2 268 931, 2 269 170, 2 269 590, 2 271 774, 2 292 144, 2 293 168, 2 293 169, and 2 302 689.

Specific neurokinin-1 receptor antagonists of use in the present invention include:

(±)-(2R3R,2S3S)-N-{[2-cyclopropoxy-5-(trifluoromethoxy)-phenyl]methyl}-2-phenylpiperidin-3-amine;

2-(S)-(3,5-bis(trifluoromethyl)benzyloxy)-3(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine;

2-(S)-(3,5-bis(trifluoromethyl)benzyloxy)-4-(3-(5-oxo-1H,4H-1,2,4-triazolo)methyl)-3-(S)-phenyl-morpholine;

2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(5-(N,N-dimethylamino)methyl-1,2,3-triazol-4-yl)methyl-3-(S)-phenylmorpholine;

2-(R)-(1-(R)-(3,5-bis(trifluoromethyl)phenyl)ethoxy)-4-(5-(N,N-dimethylamino)methyl-1,2,3-triazol-4-yl)methyl-3-(S)-(4-fluorophenyl)morpholine;

(3S,5R,6S)-3-[2-cyclopropoxy-5-(trifluoromethoxy)phenyl]-6-phenyl-1-oxa-7-aza-spiro[4.5]decane;

(3R,5R,6S)-3-[2-cyclopropoxy-5-(trifluoromethoxy)phenyl]-6-phenyl-1-oxa-7-aza-spiro[4.5]decane;

2-(R)-(1-(S)-(3,5-bis(trifluoromethyl)phenyl)-2-hydroxyethoxy)-3-(S)-(4-fluorophenyl)-4-(1,2,4-triazole-3-yl)methylmorpholine;

2-(R)-(12-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;

2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;

2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine; or a pharmaceutically acceptable salt thereof.

Suitable atypical anti-depressants include: bupropion, lithium, nefazodone, trazodone and viloxazine, and pharmaceutically acceptable salts thereof.

Suitable classes of anti-anxiety agents include benzodiazepines and 5-HT_{1A} agonists or antagonists, especially 5-H_{1A} partial agonists, and corticotropin releasing factor (CRF) antagonists.

Suitable benzodiazepines include: alprazolam, chlordiazepoxide, clonazepam, chlorazepate, diazepam, halazepam, lorazepam, oxazepam and prazepam, and pharmaceutically acceptable salts thereof.

Suitable 5-HT_{1A} receptor agonists or antagonists include, in particular, the 5-HT_{1A} receptor partial agonists buspirone, flesinoxan, gepirone and ipsapirone, and pharmaceutically acceptable salts thereof.

Suitable corticotropin releasing factor (CRF) antagonists include those previously discussed herein.

As used herein, the term “substance abuse disorders” includes substance dependence or abuse with or without physiological dependence. The substances associated with these disorders are: alcohol, amphetamines (or amphetamine-like substances), caffeine, cannabis, cocaine, hallucinogens, inhalants, marijuana, nicotine, opioids, phencyclidine (or phencyclidine-like compounds), sedative-hypnotics or benzodiazepines, and other (or unknown) substances and combinations of all of the above.

In particular, the term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances. It will be appreciated that reference to treatment of nicotine withdrawal includes the treatment of symptoms associated with smoking cessation.

Other “substance abuse disorders” include substance-induced anxiety disorder with onset during withdrawal; substance-induced mood disorder with onset during withdrawal; and substance-induced sleep disorder with onset during withdrawal.

It will be appreciated that a combination of a conventional antipsychotic drug with a CB1 receptor modulator may provide an enhanced effect in the treatment of mania. Such a combination would be expected to provide for a rapid onset of action to treat a manic episode thereby enabling prescription on an “as needed basis”. Furthermore, such a combination may enable a lower dose of the antispychotic agent to be used without compromising the efficacy of the antipsychotic agent, thereby minimizing the risk of adverse side-effects. A yet further advantage of such a combination is that, due to the action of the CB1 receptor modulator, adverse side-effects caused by the antipsychotic agent such as acute dystonias, dyskinesias, akathesia and tremor may be reduced or prevented.

Thus, according to a further aspect of the present invention there is provided the use of a CB1 receptor modulator and an antipsychotic agent for the manufacture of a medicament for the treatment or prevention of mania.

The present invention also provides a method for the treatment or prevention of mania, which method comprises administration to a patient in need of such treatment or at risk of developing mania of an amount of a CB1 receptor modulator and an amount of an antipsychotic agent, such that together they give effective relief.

In a further aspect of the present invention, there is provided a pharmaceutical composition comprising a CB1 receptor modulator and an antipsychotic agent, together with at least one pharmaceutically acceptable carrier or excipient.

It will be appreciated that the CB1 receptor modulator and the antipsychotic agent may be present as a combined preparation for simultaneous, separate or sequential use for the treatment or prevention of mania. Such combined preparations may be, for example, in the form of a twin pack.

In a further or alternative aspect of the present invention, there is therefore provided a product comprising a CB1 receptor modulator and an antipsychotic agent as a combined preparation for simultaneous, separate or sequential use in the treatment or prevention of mania.

It will be appreciated that when using a combination of the present invention, the CB1 receptor modulator and the antipsychotic agent may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, the antipsychotic agent may be administered as a tablet and then, within a reasonable period of time, the CB1 receptor modulator may be administered either as an oral dosage form such as a tablet or a fast-dissolving oral dosage form. By a “fast-dissolving oral formulation” is meant, an oral delivery form which when placed on the tongue of a patient, dissolves within about 10 seconds.

Included within the scope of the present invention is the use of CB1 receptor modulators in combination with an antipsychotic agent in the treatment or prevention of hypomania.

It will be appreciated that a combination of a conventional antipsychotic drug with a CB1 receptor modulator may provide an enhanced effect in the treatment of schizophrenic disorders. Such a combination would be expected to provide for a rapid onset of action to treat schizophrenic symptoms thereby enabling prescription on an “as needed basis”. Furthermore, such a combination may enable a lower dose of the CNS agent to be used without compromising the efficacy of the antipsychotic agent, thereby minimizing the risk of adverse side-effects. A yet further advantage of such a combination is that, due to the action of the CB1 receptor modulator, adverse side-effects caused by the antipsychotic agent such as acute dystonias, dyskinesias, akathesia and tremor may be reduced or prevented.

As used herein, the term “schizophrenic disorders” includes paranoid, disorganized, catatonic, undifferentiated and residual schizophrenia; schizophreniform disorder; schizoaffective disorder; delusional disorder; brief psychotic disorder; shared psychotic disorder; substance-induced psychotic disorder; and psychotic disorder not otherwise specified.

Other conditions commonly associated with schizophrenic disorders include self-injurious behavior (e.g. Lesch-Nyhan syndrome) and suicidal gestures.

Suitable antipsychotic agents of use in combination with a CB1 receptor modulator include the phenothiazine, thioxanthene, heterocyclic dibenzazepine, butyrophenone, diphenylbutylpiperidine and indolone classes of antipsychotic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. Suitable examples of dibenzazepines include clozapine and olanzapine. An example of a butyrophenone is haloperidol. An example of a diphenylbutylpiperidine is pimozide. An example of an indolone is molindolone. Other antipsychotic agents include loxapine, sulpiride and risperidone. It will be appreciated that the antipsychotic agents when used in combination with a CB1 receptor modulator may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, olanzapine, haloperidol, pimozide and risperidone are commonly used in a non-salt form.

Other classes of antipsychotic agent of use in combination with a CB1 receptor modulator include dopamine receptor antagonists, especially D2, D3 and D4 dopamine receptor antagonists, and muscarinic ml receptor agonists. An example of a D3 dopamine receptor antagonist is the compound PNU-99194A. An example of a D4 dopamine receptor antagonist is PNU-101387. An example of a muscarinic ml receptor agonist is xanomeline.

Another class of antipsychotic agent of use in combination with a CB1 receptor modulator is the 5-HT_{2A} receptor antagonists, examples of which include MDL100907 and fananserin. Also of use in combination with a CB1 receptor modulator are the serotonin dopamine antagonists (SDAs) which are believed to combine 5-HT_{2A} and dopamine receptor antagonist activity, examples of which include olanzapine and ziperasidone.

It will be appreciated that a combination of a conventional anti-asthmatic drug with a CB1 receptor modulator may provide an enhanced effect in the treatment of asthma.

Thus, according to a further aspect of the present invention there is provided the use of a CB1 receptor modulator and an anti-asthmatic agent for the manufacture of a medicament for the treatment or prevention of asthma.

The present invention also provides a method for the treatment or prevention of asthma, which method comprises administration to a patient in need of such treatment an amount of a compound of the present invention and an amount of an anti-asthmatic agent, such that together they give effective relief.

Suitable anti-asthmatic agents of use in combination with a compound of the present invention include, but are not limited to: (a) VLA-4 antagonists such as natalizumab and the compounds described in U.S. Pat. No. 5,510,332, WO 97/03094, WO 97/02289, WO 96/40781, WO 96/22966, WO 96/20216, WO 96/01644, WO 96/06108, WO 95/15973 and WO 96/31206; (b) steroids and corticosteroids such as beclomethasone, methylprednisolone, betamethasone, prednisone, dexamethasone, and hydrocortisone; (c) antihistamines (Hl-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (d) non-steroidal anti-asthmatics including β2-agonists (such as terbutaline, metaproterenol, fenoterol, isoetharine, albuterol, bitolterol, salmeterol, epinephrine, and pirbuterol), theophylline, cromolyn sodium, atropine, ipratropium bromide, leukotriene antagonists (such as zafirlukast, montelukast, pranlukast, iralukast, pobilukast, and SKB-106,203), and leukotriene biosynthesis inhibitors (such as zileuton and BAY-1005); (e) anti-cholinergic agents including muscarinic antagonists (such as ipratropium bromide and atropine); and (f) antagonists of the chemokine receptors, especially CCR-3; and pharmaceutically acceptable salts thereof.

It will be appreciated that a combination of a conventional anti-constipation drug with a CB1 receptor modulator may provide an enhanced effect in the treatment of constipation.

Thus, according to a further aspect of the present invention there is provided the use of a CB1 receptor modulator and an anti-constipation agent for the manufacture of a medicament for the treatment or prevention of constipation.

The present invention also provides a method for the treatment or prevention of constipation, which method comprises administration to a patient in need of such treatment an amount of a compound of the present invention and an amount of an anti-constipation agent, such that together they give effective relief.

It will be appreciated that a combination of a conventional anti-constipation drug with a CB1 receptor modulator may provide an enhanced effect in the treatment of chronic intestinal pseudo-obstruction.

Thus, according to a further aspect of the present invention there is provided the use of a CB1 receptor modulator and an anti-constipation agent for the manufacture of a medicament for the treatment or prevention of chronic intestinal pseudo-obstruction.

The present invention also provides a method for the treatment or prevention of chronic intestinal pseudo-obstruction, which method comprises administration to a patient in need of such treatment an amount of a compound of the present invention and an amount of an anti-constipation agent, such that together they give effective relief.

Suitable anti-constipation agents of use in combination with a compound of the present invention include, but are not limited to, osmotic agents, laxatives and detergent laxatives (or wetting agents), bulking agents, and stimulants; and pharmaceutically acceptable salts thereof.

A particularly suitable class of osmotic agents include, but are not limited to sorbitol, lactulose, polyethylene glycol, magnesium, phosphate, and sulfate; and pharmaceutically acceptable salts thereof.

A particularly suitable class of laxatives and detergent laxatives, include, but are not limited to, magnesium, and docusate sodium; and pharmaceutically acceptable salts thereof.

A particularly suitable class of bulking agents include, but are not limited to, psyllium, methylcellulose, and calcium polycarbophil; and pharmaceutically acceptable salts thereof.

A particularly suitable class of stimulants include, but are not limited to, anthroquinones, and phenolphthalein; and pharmaceutically acceptable salts thereof.

It will be appreciated that a combination of a conventional anti-cirrhosis drug with a CB1 receptor modulator may provide an enhanced effect in the treatment of cirrhosis of the liver.

Thus, according to a further aspect of the present invention there is provided the use of a CB1 receptor modulator and an anti-cirrhosis agent for the manufacture of a medicament for the treatment or prevention of cirrhosis of the liver.

The present invention also provides a method for the treatment or prevention of cirrhosis of the liver, which method comprises administration to a patient in need of such treatment an amount of a compound of the present invention and an anti-cirrhosis agent, such that together they give effective relief.

Suitable anti-cirrhosis agents of use in combination with a compound of the present invention include, but are not limited to, corticosteroids, penicillamine, colchicine, interferon-γ, 2-oxoglutarate analogs, prostaglandin analogs, and other anti-inflammatory drugs and antimetabolites such as azathioprine, methotrexate, leflunamide, indomethacin, naproxen, and 6-mercaptopurine; and pharmaceutically acceptable salts thereof.

The method of treatment of this invention comprises a method of modulating the CB1 receptor and treating CB1 receptor mediated diseases by administering to a patient in need of such treatment a non-toxic therapeutically effective amount of a compound of this invention that selectively antagonizes the CB1 receptor in preference to the other CB or G-protein coupled receptors.

The term “therapeutically effective amount” means the amount the compound of structural formula I that will elicit the biological or medical response of a tissue, system, animal or human that is being sought by the researcher, veterinarian, medical doctor or other clinician.

The weight ratio of the compound of the Formula I to the second active ingredient may be varied and will depend upon the effective dose of each ingredient. Generally, an effective dose of each will be used. Thus, for example, when a compound of the Formula I is combined with a β-3 agonist the weight ratio of the compound of the Formula I to the β-3 agonist will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

Abbreviations used in the following Schemes and Examples:

4-DMAP: 4-dimethylaminopyridine

Ac₂O: acetic anhydride

AcCN: acetonitrile

Ag₂O: silver(I) oxide

AIBN: 2,2′-azobisisobutyronitrile

AlMe₃: trimethylaluminum

BF₃-Et₂O: borontrifluoride etherate

BH₃-DMS: borane dimethylsulfide complex

BINAP: 2,2′-bis(diphenylphosphino)-1,1′-binaphthyl

Bn: benzyl

BOC: tert-butoxycarbonyl

BOC-ON: 2-(tert-butoxycarbonyloxyimino)-2-phenylacetonitrile

BOP: benzotriazol-1-yloxy-tris (dimethylamino)-phosphonium hexafluorophosphate

brine: saturated sodium chloride solution

CBZ: benzyloxycarbonyl

(CF₃SO₂)₂NPh: N,N-di(trifluoromethylsulfonyl)aniline

CH₂Cl₂: dichloromethane

(CH₃)₃O-BF3: trimeth yloxoni um fluoroborate

CO: carbon monoxide

Cy₃P: tricyclohexylphosphine

DBU: 1,8-diazobicyclo[5.4.0]undec-7-ene

DCC: dicyclohexylcarbodiimide

DIBAL-H: diisobutylaluminum hydride

DIPEA: N,N-diisopropylethylamine

DME: 1,2-dimethoxyethane

DMF: dimethylformamide

DMPU: 1,3-dimethyl-3,4,5,6-tetrahydro-2(1H)-pyrimidinone

DMSO: dimethylsulfoxide

DPPF: 1,1′-bis(diphenylphosphino)ferrocene

EDC: 1-ethyl-3-(3-dimethylaminopropyl)-carbodiimide hydrochloride

Et: ethyl

Et₂O: diethyl ether

EtOAc: ethyl acetate

EtOH: ethanol

Et₃N: triethyl amine

FMOC: 9-fluorenylmethoxylcarbonyl

g or gm: gram

h or hr: hours

HATU: O-(7-azabenzotriazol-1-yl)-1,1,3,3-tetramethyluronium hexafluorophosphate

HBTU: O-(benzotriazol-1-yl)-1,1,3,3-tetramethyluronium hexafluorophosphate

HOAc: acetic acid

HOAt: 1-hydroxy-7-azabenzotriazole

HOBt: 1-hydroxybenzotriazole

HPLC: high pressure liquid chromatography

H₂SO₄ sulfuric acid

in vacuo: rotoevaporation

KOAc: potassium acetate

LC-MS liquid chromatography-mass spectrum

LDA: lithium diisopropylamide

LiHMDS: lithium hexamethyldisilylamide

mCPBA: meta-chloroperbenzoic acid

Me: methyl

MeI: methyl iodide

MeOH or CH₃OH: methanol

mg: milligram

MHz: megahertz

min: minutes

mL: milliliter

mmol: millimole

MPLC: medium pressure liquid chromatography

MS or ms: mass spectrum

MsCl: methanesulfonyl chloride

Na₂CO₃: sodium carbonate

NaNO₂: sodium nitrite

NaOAc: sodium acetate

Na(OAc)₃BH: sodium triacetoxy borohydride

NBS: N-bromosuccinimide

NH₄OAc: ammonium acetate

NMO: 4-methyl-morpholine-N-oxide

Pd₂dba₃: tris(dibenzylideneacetone)dipalladium(0)

Pd(OAc)₂: palladium acetate

Ph: phenyl

Ph₃P: triphenylphosphine

POCl₃: phosphoryl trichloride

pTSA: para-toluenesulfonic acid

PyBOP: (benzotriazol-1-yloxy)tripyrrolidinophosphonium hexafluorophosphate

rt: room temperature

TBAF: tetrabutylammonium fluoride

TBSCl: tert-butyldimethylsilyl chloride

t-Bu₃P: tri-tert-butylphosphine

TEA: triethylamine

TFA: trifluoroacetic acid

THF: THF

TLC: thin layer chromatography

TMSCHN₂: trimethylsiliyldiazomethane

TMSCl: trimethylsilyl chloride

TMSI: trimethylsilyl iodide

TPAP: tetrapropylammonium perruthenate

aTsCl: para-toluene sulfonyl chloride

TsOH: para-toluene sulfonic acid

Zn: zinc

Compounds of the present invention may be prepared by procedures illustrated in the accompanying schemes.

As outlined in Scheme 1, a β-keto ester A is reacted with sodium nitrite in aqueous acetic acid to form the α-oxime B. Reduction of the oxime in B with zinc in the presence of sulfuric acid followed by reaction with an acyl chloride affords α-amido-β-keto ester C. An aniline is reacted with C in the presence of a strong acid in refluxing xylenes to produce diaryl-imidazole ester D. The ester in D is transformed into a variety of amides by reaction with amines in the presence of trialkylaluminum to yield diaryl-imidazole amides E.

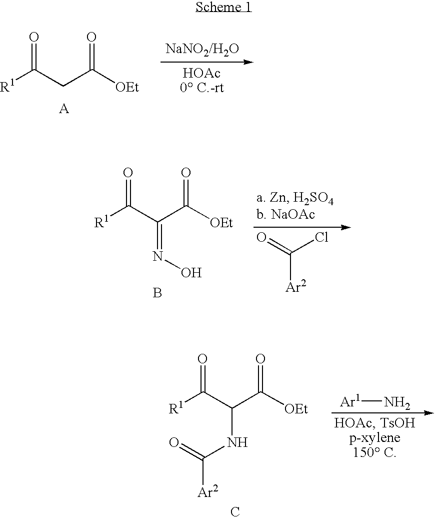

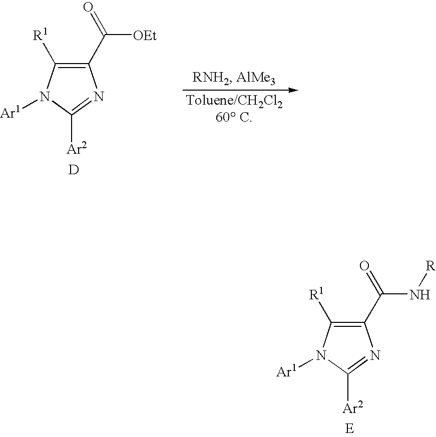

Alternatively, as shown in Scheme 2, an α-keto ester A is reacted with an aniline in the presence of a reducing agent to form N-aryl α-amino acid ester B. The amine in B is acylated with an aroyl chloride to form C which is cyclized in the presence of ammonium acetate to form imidazolinone D.

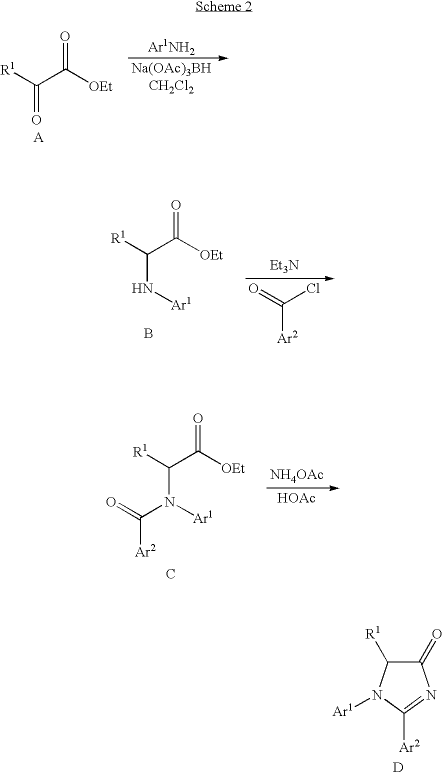

Alternatively, as shown in Scheme 3, B is reacted with imino ether F to form D directly. The imino ether F is prepared from an aryl nitrile reacted with methanol in the presence of strong acid or from an aryl amide by reacting with trimethyloxonium fluoroborate.

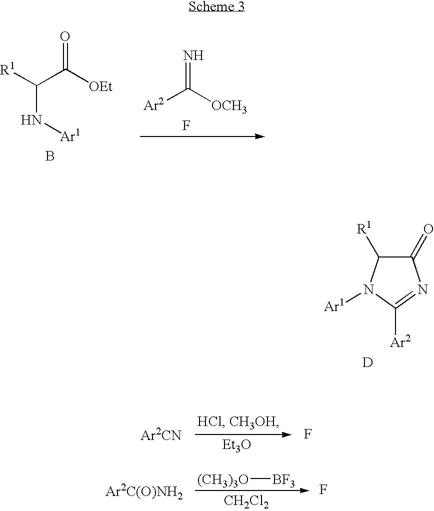

As shown in Scheme 4, the imidazolinone D is reacted with base and an alkylating agent to form the ether E. Reaction of D with phosphoryl trichloride forms G (X=Cl). Alternatively, D is reacted with N,N-di(trifluoromethyl-sulfonyl)aniline in the presence an amine base to form the triflate G (X=OSO₂CF₃). Reaction of G with an amine in the presence of palladium acetate and a sterically hindered phosphine ligand affords 4-amino-imidazole H. Reaction of G with an alcohol or an amine in the presence of palladium acetate, carbon monoxide, and DPPF yields ester or amide I, respectively.

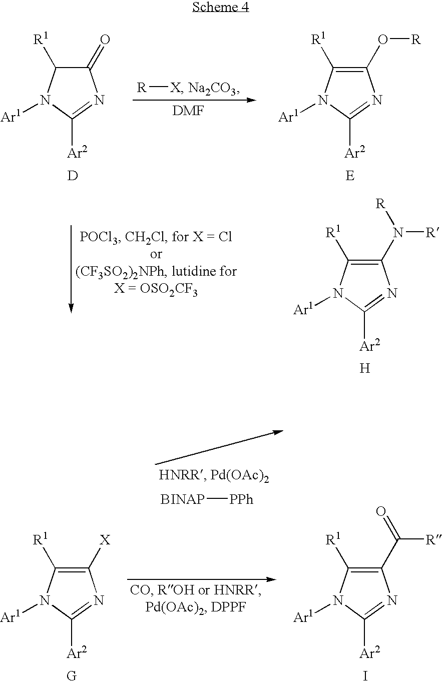

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

General Procedures.

The LC/MS analyses were preformed using a Micromass ZMD mass spectrometer coupled to an Agilent 1100 Series HPLC utilizing a YMC ODS-A 4.6×50 mm column eluting at 2.5 mL/min with a solvent gradient of 10 to 95% B over 4.5 min, followed by 0.5 min at 95% B: solvent A=0.06% TFA in water; solvent B=0.05% TFA in acetonitrile. ¹H-NMR spectra were obtained on a 500 MHz Varian Spectrometer in CDCl₃ or CD₃OD as indicated and chemical shifts are reported as δ using the solvent peak as reference and coupling constants are reported in hertz (Hz).

### EXAMPLE 1

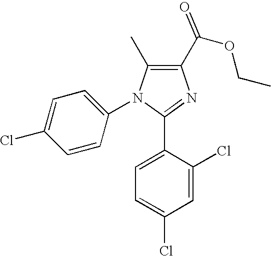

### Ethyl 1-(4-chlorophenyl)-2-(2.4-dichlorophenyl)-5-methyl-imidazole-4-carboxylate

Step A: Ethyl 2-(2,4-dichlorobenzoyl)amino-3-oxobutyrate.

To a solution prepared by mixing 19 mL of 30% (w/v) H₂SO₄ with 21 g of ice 1.987 g (12.49 mmol) of ethyl 2-(hydroxyimino)-3-oxobutyrate (prepared as described in J. Chem. Soc., Perkin Trans. 1991, 881-891) was added and the mixture was cooled in an ice bath. To this solution 2.45 g (37.5 mmol) of Zn dust was added in portions. The reaction was stirred for 0.5 hr in an ice bath after all the Zn was added, then the solution was filtered through a pad of celite and the solids were rinsed with water. The filtrate was treated with 12.92 g (94.9 mmol) of NaOAc and 1.75 mL (12.49 mmol) of 2,4-dichlorobenzoyl chloride in 20 mL of CH₂Cl₂ with cooling in ice bath. The bath was removed and the reaction was stirred overnight. The mixture was extracted with CH₂Cl₂ three times. The combined extracts were washed with saturated NaHCO₃ and brine, dried and concentrated. The residue was chromatographed on a flash column using a gradient of 10-20% EtOAC/hexane to isolate the desired product.

¹H NMR: (500 MHz, CDCl₃): δ 1.38 (t, 3H) 2.50 (s, 3H), 4.36 (m, 2H), 5.44 (d, 1H), 7.3-7.8 (m, 4H).

Step B: Ethyl 1-(4-chlorophenyl)-2-(2,4-dichlorophenyl)-5-methyl-imidazole-4-carboxylate.

To a solution of 0.506 g (1.59 mmol) of ethyl 2-(2,4-dichlorobenzoyl)amino-3-oxobutyrate in 5 mL of p-xylene and 1 mL of acetic acid, 0.223 g (1.75 mmol) of 4-chloroaniline was added. The flask was fitted with a Dean Stark trap and the mixture was heated to 150° C. to remove water. After 2 hr a few crystals of TsOH were added, and the heating was continued overnight. The reaction was cooled, concentrated, and the residue was partitioned between EtOAc and saturated NaHCO₃. The organic layer was washed with brine, dried and concentrated. The residue was purified on a flash column with a gradient of 20-30% EtOAc/hexane to obtain the title compound.

¹H NMR: (500 MHz, CDCl₃): δ 1.45 (t, 3H), 2.48 (s, 3H), 4.46 (q, 2H), 7.0-7.5 (m, 7H). LC-MS: Rₜ=3.66 min. m/e=409 (M+1).

### EXAMPLE 2

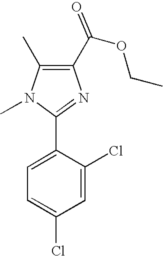

### Ethyl 2-(2,4-dichlorophenyl)-1.5-dimethyl-imidazole4-carboxylate

A solution of 99 mg (0.31 mmol) of ethyl 2-(2,4-dichlorobenzoyl)amino-3-oxobutyrate in 2 mL of p-xylene and 0.5 mL of acetic acid was treated with 1.6 mL (3.2 mmol) of 2M methylamine in THF. The flask was fitted with a Dean Stark trap and the solution heated to reflux to remove water. After 1 hr the reaction was cooled, diluted with EtOAc, washed with saturated NaHCO₃, brine and dried. The filtrate was concentrated, and the residue was purified by prep TLC using 30% EtOAc/hexane to furnish the title compound.

¹H NMR: (500 MHz, CDCl₁₃): δ 1.42 (t, 3H), 2.63 (s, 3H), 3.43 (s, 3H), 4.41 (q, 2H), 7.3-7.6 (m, 3H). LC-MS: Rₜ=2.3 min. m/e=313 (M+1).

### EXAMPLE 3

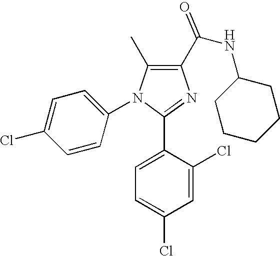

### N-(Cyclohexyl)-1-(4-chlorophenyl)-2-(2.4-dichlorolphenyl)-5-methyl-imidazole-4-carboxamide

To a solution of 0.1 mL (0.9 mmol) of cyclohexylamine in 0.5 mL of dry toluene under N₂, 0.45 mL (0.9 mmol) of 2M trimethylaluminium in hexane was added with cooling in ice bath. The cold bath was removed, reaction was stirred for 1 hr and 0. 148 g (0.36 mmol) of ethyl 1-(4-chlorophenyl)-2-(2,4-dichlorophenyl)-5-methyl-imidazole-4-carboxylate in 1 mL of toluene and 0.5 mL of CH₂Cl₂ was added. The mixture was heated in a 60° C. bath for 2 hr, cooled, quenched with water and the pH was adjusted to 5 with 1.2 N HCl. The solution was extracted with EtOAc three times. The combined EtOAc layer was washed with brine, dried and concentrated. The residue was purified by prep TLC using 30% EtOAc/hexane as an eluant to isolate the title compound.

¹H NMR: (500 MHz, CDCl₃): δ 1.2-2.1 (m, 1H), 2.51 (s, 3H), 3.98 (m, 1H), 6.64 (s, 1H), 7.0-7.5 (m, 7H). LC-MS: Rₜ=4.4 min. m/e=464 (M+1).

### EXAMPLE 4

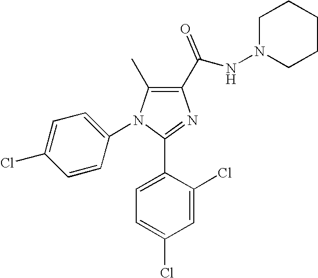

### N-(Piperidin-1-yl)-1-(4-chlorophenyl)-2-(2,4-dichlorophenyl)-5-methyl-imidazole-4-carboxamide

The title compound was synthesized by the method described in Example 3 but substituting 1-aminopiperidine for cyclohexylamine.

LC-MS: Rₜ=3.12 min. m/e=463 (M+1).

### EXAMPLE 5

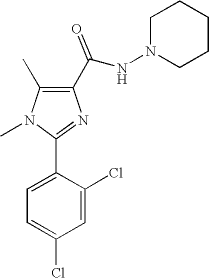

### N-(Piperidin-1-yl)-2-(2,4-dichlorophenyl)-1,5-dimethyl-imidazole-4-carboxamide

The title compound was prepared from ethyl 2-(2,4-dichlorophenyl)-1,5-dimethyl-imidazole-4-carboxylate following the method described in Example 3 but substituting 1-aminopiperidine for cyclohexylamine.

LC-MS: Rₜ=2.17 min. m/e=367 (M+1).

The following compounds were prepared by the procedures described in Example 3 by substituting the appropriate amine for cyclohexylamine.

### EXAMPLE 6

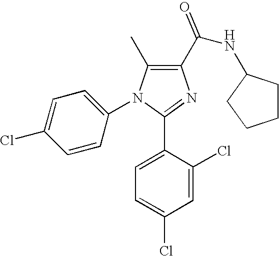

### N-(Cyclopentyl)-1-(4-chlorophenyl)-2-(2.4-dichlorophenyl)-5-methyl-imidazole-4-carboxamide

LC-MS: Rₜ=4.2 min. m/e=450.2 (M+1).

### EXAMPLE 7

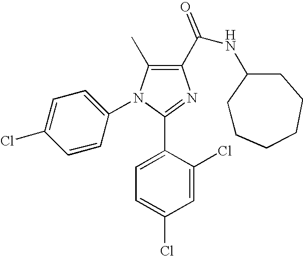

### N-(Cycloheptyl)-1-(4-chlorophenyl)-2-(2,4-dichloropohenyl)-5-methyl-imidazole-4-carboxamide

LC-MS: Rₜ=4.5 min. m/e=478.2 (M+1).

### EXAMPLE 8

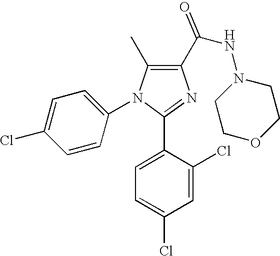

### N-(Morpholin-4-yl)-1-(4-chlorophenyl)-2-(2,4-dichlorophenyl)-5-methyl-imidazole-4-carboxamide

LC-MS: Rₜ=: 3.3 min. m/e=465.1 (M+1).

### EXAMPLE 9

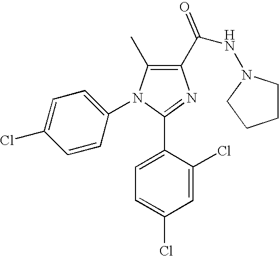

### N-(Pyrrolidin-1-yl)-1-(4-chlorophenyl)-2-(2,4-dichlorophenyl)-5-methyl-imidazole-4-carboxamide

LC-MS: Rₜ=2.96 min. m/e=449.2 (M+1).

### EXAMPLE 10

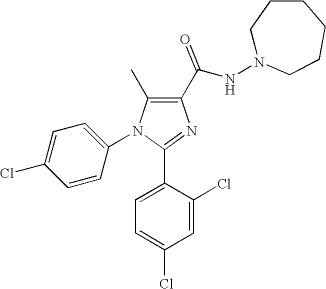

### N-(Azepin-1-yl)-1-(4-chlorophenyl)-2-(2,4-dichlorophenyl)-5-methyl-imidazole-4-carboxamide

LC-MS: Rₜ=3.2 min. m/e=477.2 (M+1).

### EXAMPLE 11

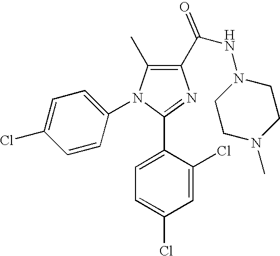

### N-(4-Methylpiperazin-1-yl)-1-(4-chlorophenyl)-2-(2,4-dichlorophenyl)-5-methyl-imidazole-4-carboxamide

LC-MS: Rₜ=2.7 min. m/e=480.1 (M+1).

### EXAMPLE 12

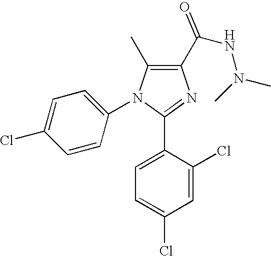

### N′,N′-Dimethyl-1-(4-chlorophenyl)-2-(2.4-dich lorophenyl)-5-methyl-imidazole-4-carboxhydrazide

LC-MS: Rₜ=2.9 min. m/e=423.1 (M+1).

The following compounds were prepared by the procedures described in Example 1 by substituting the appropriate aroylchloride for 2,4-dichlorobenzoyl chloride in Step A and the appropriate aniline derivative for 4-chloroaniline in Step B. The various amides were prepared according to the procedure described in Example 3 by substituting the appropriate amine for cyclohexylamine.

### EXAMPLE 13

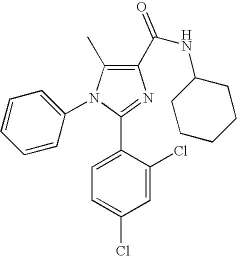

### N-(Cyclohexyl)-1-(phenyl)-2-(2.4-dichlorophenyl)-5-methyl-imidazole-4-carboxamide

LC-MS: Rₜ=4.1 min. m/e=428.1 (M⁺).

### EXAMPLE 14

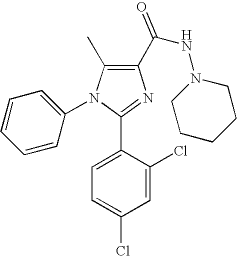

### N-(Piperidin-1-yl)-1-(phenyl)-2-(2,4-dichlorophenyl)-5-methyl-imidazole-4-carboxamide

LC-MS: Rₜ=2.8 min. m/e=429.0 (M+).

### EXAMPLE 15

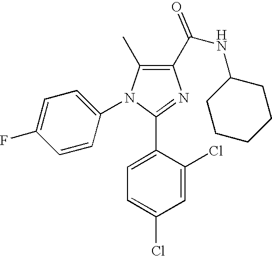

### N-(Cyclohexyl)-1-(4-fluorophenyl)-2-(2,4-dichlorophenyl)-5-methyl-imidazole-4-carboxamide

LC-MS: Rₜ=4.1 min. m/e=446.0 (M⁺).

### EXAMPLE 16

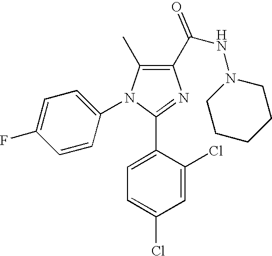

### N-(Piperidin-1-yl)-1-(4-fluorophenyl)-2-(2 4-dichlorophenyl)-5-methyl-imidazole-4-carboxamide

LC-MS: Rₜ=2.9 min. m/e=447.0 (M⁺).

### EXAMPLE 17

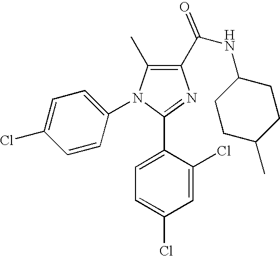

### N-(4-Methyl-cyclohexyl)-1-(4-chlorophenyl)-2-(2,4-dichlorophenyl)-5-methyl-imidazole-4-carboxamide (Isomer A)

LC-MS: Rₜ=4.6 min. m/e=476.2 (M⁺).

### EXAMPLE 18

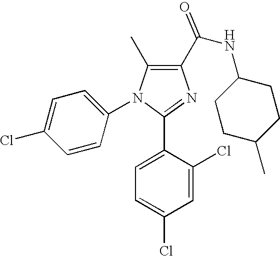

### N-(4-Methyl-cyclohexyl)-1-(4-chlorophenyl)-2-(2.4-dichlorophenyl)-5-methyl-imidazole-4-carboxamide (Isomer B)

LC-MS: Rₜ=4.6 min. m/e=476.2 (M⁺).

### EXAMPLE 19

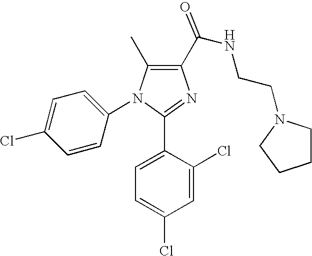

### N-(2-(Pyrrolidin-1-yl)ethyl)-1-(4-chlorophenyl)-2-(2,4-dichlorophenyl)-5-methyl-imidazole-4-carboxamide

LC-MS: Rₜ=2.9 min. m/e=477.1 (M⁺).

### EXAMPLE 20

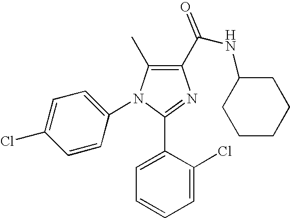

### N-(Cyclohexyl)-1-(4-chlorophenyl)-2-(2-chlorophenyl)-5-methyl-imidazole-4-carboxamide

LC-MS: Rₜ=4.0 min. m/e=428.2 (M⁺).

### EXAMPLE 21

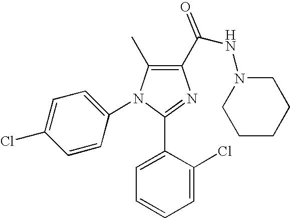

### N-(Piperidin-1-yl)-1-(4-chlorophenyl)-2-(2-chlorophenyl)-5-methyl-imidazole-4-carboxamide

LC-MS: Rₜ=2.8 min. m/e=429.2 (M⁺).

### EXAMPLE 22

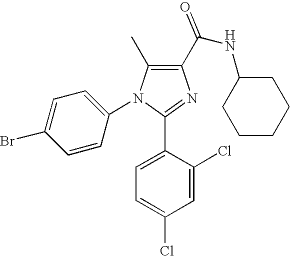

### N-(Cyclohexyl)-1-(4-bromophenyl)-2-(2,4-dichlorophenyl)-5-methyl-imidazole-4-carboxamide

LC-MS: Rₜ=4.4 min. m/e=508.0 (M⁺).

### EXAMPLE 23

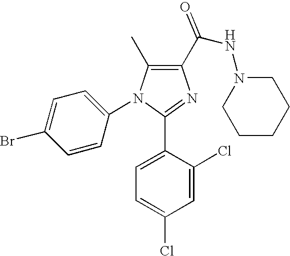

### N-(Piperidin-1-yl)-1-(4-bromophenyl)-2-(2,4-dichlorophenyl)-5-methyl-imidazole-4-carboxamide

LC-MS: Rₜ=3.3 min. m/e=509.1 (M⁺).

### EXAMPLE 24

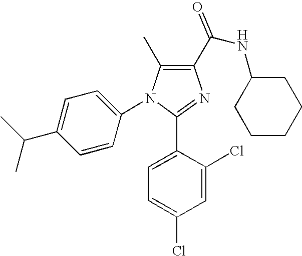

### N-(Cyclohexyl)-1-(4-isopropylphenyl)-2-(2,4-dichlorophenyl)-5-methyl-imidazole-4-carboxamide

LC-MS: Rₜ=4.6 min. m/e=470.2 (M⁺).

### EXAMPLE 25

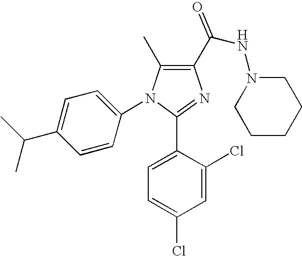

### N-(Piperidin-1-yl)-1-(4-isopropylphenyl)-2-(2,4-dichlorophenyl)-5-methyl-imidazole-4-carboxamide

LC-MS: Rₜ=3.4 mn. m/e=471.2 (M⁺).

### EXAMPLE 26

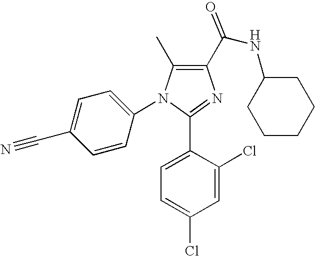

### N-(Cyclohexyl)-1-(4-cyanophenyl)-2-(2,4-dichlorophenyl)-5-methyl-imidazole-4-carboxamide

LC-MS: Rₜ=4.0 min. m/e=453.1 (M⁺).

### EXAMPLE 27

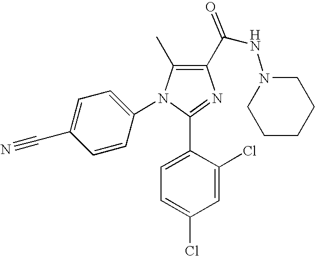

### N-(Piperidin-1-yl)-1-(4-cyanophenyl)-2-(2,4-dichlorophenyl)-5-methyl-imidazole-4-carboxamide

LC-MS: Rₜ=2.2 min. m/e=471.1 (M⁺).

### EXAMPLE 28

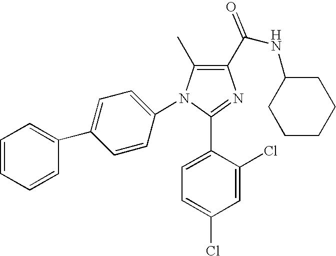

### N-(Cyclohexyl)-1-(4-biphenyl)-2-(2,4-dichlorophenyl)-5-methyl-imidazole-4-carboxamide

LC-MS: Rₜ=4.6 min. m/e=504.1 (M⁺).

### EXAMPLE 29

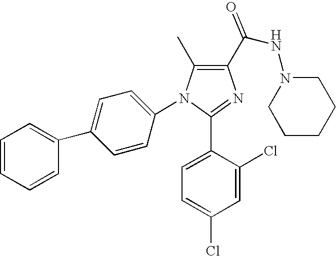

### N-(Piperidin-1-yl)-1-(4-biphenyl)-2-(2,4-dichlorophenyl)-5-methyl-imidazole-4-carboxamide

LC-MS: Rₜ=3.4 min. m/e=505.1 (M⁺).

### EXAMPLE 30

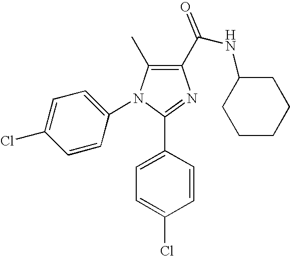

### N-(Cyclohexyl)-1,2-bis(4-chlorophenyl)-5-methyl-imidazole-4-carboxamide

LC-MS: Rₜ=4.2 min. m/e=428.2 (M⁺).

### EXAMPLE 31

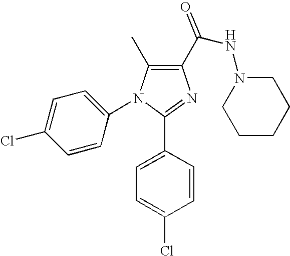

### N-(Piperidin-1-yl)-1,2-bis(4-chlorophenyl)-5-methyl-imidazole-4-carboxamide

LC-MS: Rₜ=3.1 min. m/e=429.2 (M⁺).

### EXAMPLE 32

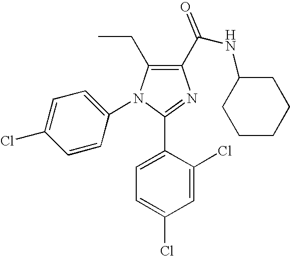

### N-(Cyclohexyl)-1-(4-chlorophenyl)-2-(2,4-dichlorophenyl)-5-ethyl-imidazole-4-carboxamide

LC-MS: Rₜ=4.6 min. m/e=476.1 (M⁺).

### EXAMPLE 33

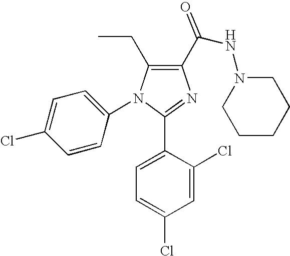

### N-(Piperidin-1-ly)-1-(4-chlorophenyl)-2-(2,4-dichlorophenyl)-5-ethyl-imidazole-4-carboxamide

LC-MS: Rₜ=3.3 min. m/e=479.1 (M⁺).

### EXAMPLE 34

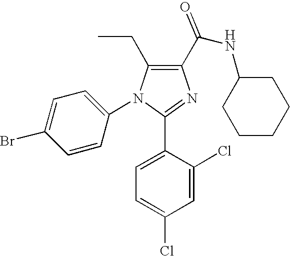

### N-(Cyclohexyl)-1-(4-bromophenyl)-2-(2,4-dichlorophenyl)-5-ethyl-imidazole-4-carboxamide

LC-MS: Rₜ=4.6 min. m/e=522.0 (M⁺).

### EXAMPLE 35

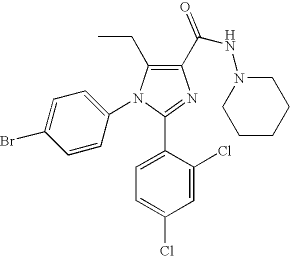

### N-(Piperidin-1-yl)-1-(4-bromophenyl)-2-(2,4-dichlorophenyl)-5-ethyl-imidazole-4-carboxamide

LC-MS: Rₜ=3.4 min. m/e=523.0 (M⁺).

### EXAMPLE 36

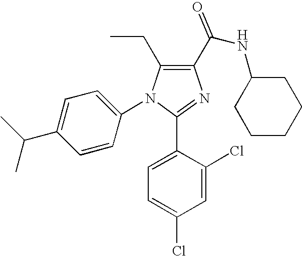

### N-(Cyclohexyl)-1-(4-isopropylphenyl)-2-(2,4-dichlorophenyl)-5-ethyl-imidazole-4-carboxamide

LC-MS: Rₜ=4.7 min. m/e 484.2 (M⁺).

### EXAMPLE 37

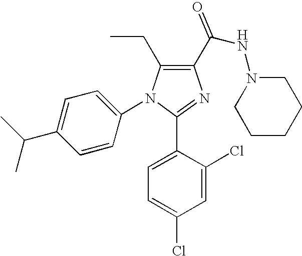

### N-(Piperidin-1-yl)-1-(4-isopropylphenyl)-2-(2,4-dichlorophenyl)-5-ethyl-imidazole-4-carboxamide

LC-MS: Rₜ=3.5 min. m/e=485.2 (M⁺).

### EXAMPLE 38

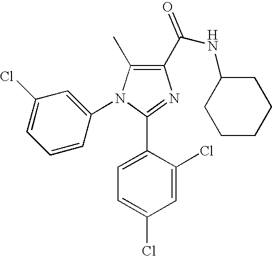

### N-(Cyclohexyl)-1-(3-chlorophenyl)-2-(2,4-dichlorophenyl)-5-methyl-imidazole-4-carboxamide

LC-MS: Rₜ=4.3 min. m/e=464.1 (M⁺).

### EXAMPLE 39

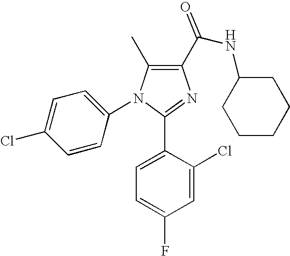

### N-(Cyclohexyl)-1-(4-chlorophenyl)-2-(2-chloro4-fluorophenyl)-5-methyl-imidazole-4-carboxamide

LC-MS: Rₜ=4.3 min. m/e=446.1 (M⁺).

### EXAMPLE 40

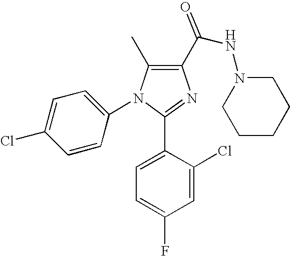

### N-(Piperidin-1-yl)-1-(4-chlorophenyl)-2-(2-chloro-4-fluorophenyl)-5-methyl-imidazole-4-carboxamide

LC-MS: Rₜ=2.9 min. m/e=447.1 (M⁺).

### EXAMPLE 41

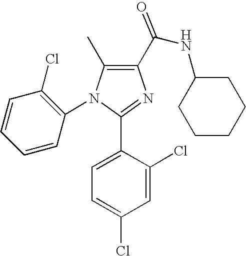

### N-(Cyclohexyl)-1-(2-chlorophenyl)-2-(2,4-dichlorophenyl)-5-methyl-imidazole-4-carboxamide

LC-MS: Rₜ=4.3 min. m/e=462.1 (M⁺).

### EXAMPLE 42

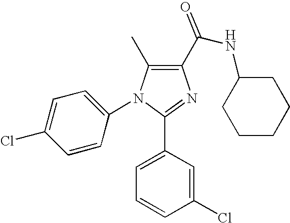

### N-(Cyclohexyl)-1-(4-chlorophenyl)-2-(3-chlorophenyl)-5-methyl-imidazole-4-carboxamide

LC-MS: Rₜ=4.3 min. m/e=428.0 (M⁺).

### EXAMPLE 43

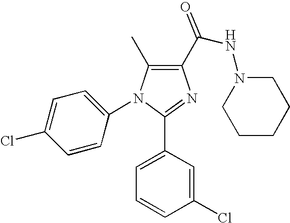

### N-(Cyclohexyl)-1-(4-chlorophenyl)-2-(3-chlorophenyl)-5-methyl-imidazole-4-carboxamide

LC-MS: Rₜ=3.0 min. m/e=429.0 (M⁺).

### EXAMPLE 43

### Cannabinoid Receptor-1 (CB1) Binding Assay.

Binding affinity determination is based on recombinant human CB1 receptor expressed in Chinese Hamster Ovary (CHO) cells (Felder et al, Mol. Pharmacol. 48: 443-450, 1995). Total assay volume is 250 μl (240 μl CB1 receptor membrane solution plus 5 μl test compound solution plus 5 μl [3H]CP-55940 solution). Final concentration of [3H]CP-55940 is 0.6 nM. Binding buffer contains 50 mM Tris-HCl, pH7.4, 2.5 mM EDTA, 5 mM MgCl_{2, 0.5} mg/ml fatty acid free bovine serum albumin and protease inhibitors (Cat#P8340, from Sigma). To initiate the binding reaction, 5 μl of radioligand solution is added, the mixture is incubated with gentle shaking on a shaker for 1.5 hours at 30° C. The binding is terminated by using 96-well harvester and filtering through GF/C filter presoaked in 0.05% polyethylenimine. The bound radiolabel is quantitated using scintillation counter. Apparent binding affinities for various compounds are calculated from IC50 values (DeBlasi et al., Trends Pharmacol Sci 10: 227-229, 1989).

The binding assay for CB2 receptor is done similarly with recombinant human CB2 receptor expressed in CHO cells.

### EXAMPLE 44

### Cannabinoid Receptor-1 (CB1) Functional Activity Assay.

The functional activation of CB1 receptor is based on recombinant human CB1 receptor expressed in CHO cells (Felder et al, Mol. Pharmacol. 48: 443-450, 1995). To determine the agonist activity or inverse agonist activity of any test compound, 50 μl of CB1-CHO cell suspension are mixed with test compound and 70 ul assay buffer containing 0.34 mM 3-isobutyl-1-methylxanthine and 5.1 μM of forskolin in 96-well plates. The assay buffer is comprised of Earle's Balanced Salt Solution supplemented with 5 mM MgCl₂. 1 mM glutamine, 10 mM HEPES, and 1 mg/ml bovine serum albumin. The mixture is incubated at room temperature for 30 minutes, and terminated by adding 30 μl/well of 0.5M HCl. The total intracellular cAMP level is quantitated using the New England Nuclear Flashplate and cAMP radioimmunoassay kit.

To determine the antagonist activity of test compound, the reaction mixture also contains 0.5 nM of the agonist CP55940, and the reversal of the CP55940 effect is quantitated. Alternatively, a series of dose response curves for CP55940 is performed with increasing concentration of the test compound in each of the dose response curves.

The functional assay for the CB2 receptor is done similarly with recombinant human CB2 receptor expressed in CHO cells.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method of treating a disease mediated by the cannabinoid-1 receptor comprising administration to a patient in need of such treatment of a therapeutically effective amount of a compound of structural formula I: 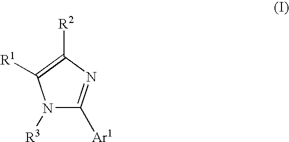
or a pharmaceutically acceptable salt thereof, wherein:
R¹ is selected from:
(1) hydrogen,
(2) C₁₋₄alkyl,
(3) C₂₋₄alkenyl,
(4) C₂₋₄alkynyl,
(5) C₃₋₇cycloalkyl,
(6) C₃₋₇cycloalkyl-C₁₋₄alkyl,
(7) cycloheteroalkyl,
(8) cycloheteroalkyl-C₁₋₄alkyl,
(9) aryl, and
(10) heteroaryl;
wherein alkyl, alkenyl, alkynyl, cycloalkyl, and cycloheteroalkyl are optionally substituted with one to four substituents independently selected from R^{a}, and aryl and heteroaryl are optionally substituted with one to four substituents independently selected from R^{b};
R² is selected from:
(1) —OR^{c},
(2) —OC(O)R^{c},
(3) —OC(O)NR^{c}R^{d},
(4) —SR^{c},
(5) —S(O)ₘR^{c},
(6) —SO₂NR^{CR}^{d},
(7) —NR^{c}R^{d},
(8) —NR^{c}C(O)R^{d},
(9) —NR^{c}SO₂R^{d},
(10) —NR^{c}C(O)NR^{c}R^{d},
(11) —NR^{c}C(O)OR^{d},
(12) —C(O)OR^{c}, and
(13) —C(O)NR^{c}R^{d};
R³ is selected from:
(1) —C₁₋₁₀alkyl, and
(2) —Ar²;
Ar¹ and Ar² are independently selected from phenyl, naphthyl, thienyl, furanyl, pyrrolyl, benzothienyl, benzofuranyl, indanyl, indenyl, indolyl, tetrahydronaphthyl, 2,3-dihydrobenzofuranyl, dihydrobenzopyranyl, and 1,4-benzodioxanyl, each optionally substituted with one, two, or three groups independently selected from R^{b};
each R^{a} is independently selected from:
(1) —OR^{c},
(2) —NR^{c}S(O)ₘR^{d},
(3) halogen,
(4) —S(O)ₘR^{c},
(5) —SR^{c},
(6) —S(O)₂OR^{c},
(7) —S(O)ₘNR^{c}R^{d},
(8) —NR^{c}R^{d},
(9) —O(CR^{e}R^{f})ₙNR^{c}R^{d},
(10) —C(O)R^{c},
(11) —CO₂R^{c},
(12) —CO₂(CR^{e}R^{f})ₙCONR^{c}R^{d},
(13) —OC(O)R^{c},
(14) —CN,
(15) —C(O)NR^{c}R^{d},
(16) —NR^{c}C(O)R^{d},
(17) —OC(O)NR^{c}R^{d},
(18) —NR^{c}C(O)OR^{d},
(19) —NR^{c}C(O)NR^{c}R^{d},
(20) —CR^{c}(N—OR^{d}),
(21) —CF₃,
(22) —OCF₃,
(23) C₃₋₈cycloalkyl, and
(24) cycloheteroalkyl;
each R^{b} is independently selected from:
(1) C₁₋₆alkyl,
(2) C₂₋₆alkenyl,
(3) C₂₋₆alkynyl,
(4) —OR^{c},
(5) —NR^{c}S(O)ₘR^{d},
(6) —NO₂,
(7) halogen,
(8) —S(O)ₘR^{c},
(9) —SR^{c},
(10) —S(O)₂OR^{c},
(11) —S(O)ₘNR^{c}R^{d},
(12) —NR^{c}R^{d},
(13) —O(CR^{e}R^{f})ₙNR^{c}R^{d},
(14) —C(O)R^{c},
(15) —CO₂R^{c},
(16) —CO₂(CR^{e}R^{f})ₙCONR^{c}R^{d},
(17) —OC(O)R^{c},
(18) —CN,
(19) —C(O)NR^{c}R^{d},
(20) —NR^{c}C(O)R^{d},
(21) —OC(O)NR^{c}R^{d},
(22) —NR^{c}C(O)OR^{d},
(23) —NR^{c}C(O)NR^{c}R^{d},
(24) —CR^{C}(N—OR^{d}),
(25) —CF₃,
(26) —OCF₃,
(27) C₃₋₈cycloalkyl,
(28) cycloheteroalkyl, and
(29) phenyl;
each R^{c} and R^{d} is independently selected from:
(1) hydrogen,
(2) C₁₋₁₀alkyl,
(3) C₂₋₁₀alkenyl,
(4) C₂₋₁₀alkynyl,
(5) —NH(C₁₋₁₀alkyl),
(6) —N(C₁₋₁₀alkyl)₂,
(7) cycloalkyl,
(8) cycloalkyl-C₁₋₁₀alkyl;
(9) cycloheteroalkyl,
(10) cycloheteroalkyl-C₁₋₁₀alkyl;
(11) aryl,
(12) heteroaryl,
(13) aryl-C₁₋₁₀alkyl, and
(14) heteroaryl-C₁₋₁₀alkyl, or
R^{c} and R^{d} together with the atom to which they are attached form a heterocyclic ring of 4 to 7 members containing 0-2 additional heteroatoms independently selected from oxygen, sulfur and N—R^{c}, each R^{c} and R^{d} may be unsubstituted or substituted with one to three substituents selected from R^{e};
each R^{e} and R^{f} is independently selected from:
(1) hydrogen,
(2) C₁₋₁₀alkyl,
(3) C₂₋₁₀alkenyl,
(4) C₂₋₁₀alkynyl,
(5) cycloalkyl,
(6) cycloalkyl-C₁₋₁₀alkyl,
(7) cycloheteroalkyl,
(8) cycloheteroalkyl-C₁₋₁₀alkyl,
(9) aryl,
(10) heteroaryl,
(11) aryl-C₁₋₁₀alkyl, and
(12) heteroaryl-C₁₋₁₀alkyl, or
R^{e} and R^{f} together with the carbon to which they are attached form a ring of 5 to 7 members containing 0-2 heteroatoms independently selected from oxygen, sulfur and nitrogen;
m is selected from 1 and 2; and
n is selected from 1, 2, and 3.

2. The method according to claim 1 wherein the disease mediated by the Cannabinoid-1 receptor is selected from: psychosis, memory deficit, cognitive disorders, migraine, neuropathy, neuro-inflammatory disorders, cerebral vascular accidents, head trauma, anxiety disorders, stress, epilepsy, Parkinson's disease, schizophrenia, substance abuse disorders, constipation, chronic intestinal pseudo-obstruction, cirrhosis of the liver, asthma, obesity, and other eating disorders associated with excessive food intake.

3. The method according to claim 2 wherein the disease mediated by the Cannabinoid-1 receptor is an eating disorder associated with excessive food intake.

4. The method according to claim 3 wherein the eating disorder asssociated with excessive food intake is selected from obesity, bulimia nervosa, and compulsive eating disorders.

5. The method according to claim 4 wherein the eating disorder associated with excessive food intake is obesity.

6. The method according to claim 1, wherein in the compound of structural formula I:
R¹ is selected from:
(1) hydrogen,
(2) C₁₋₄alkyl,
(3) C₂₋₄alkenyl,
(4) C₂₋₄alkynyl,
(5) C₃₋₇cycloalkyl, and
(6) C₃₋₇cycloalkyl-C₁₋₄alkyl,
wherein alkyl, alkenyl, alkynyl, and cycloalkyl, are optionally substituted with one to four substituents independently selected from R^{a};
R² is selected from:
(1) —OR^{c},
(2) —SR^{c},
(3) —S(O)ₘR^{c},
(4) —SO₂NR^{c}R^{d},
(5) —NR^{c}R^{d},
(6) —NR^{c}C(O)R^{d},
(7) —NR^{c}SO₂R^{d},
(8) —C(O)OR^{c}, and
(9) —C(O)NR^{c}R^{d};
R³ is selected from:
(1) —C₁₋₄alkyl, and
(2) —Ar²;
Ar¹ and Ar² are independently selected from phenyl, naphthyl, thienyl, each optionally substituted with one or two groups independently selected from R^{b};
each R^{a} is independently selected from:
(1) —OR^{c},
(2) —NR^{c}S(O)ₘR^{d},
(3) halogen,
(4) —S(O)ₘR^{c},
(5) —SR^{c},
(6) —S(O)ₘNR^{c}R^{d},
(7) —NR^{c}R^{d},
(8) —O(CR^{e}R^{f})ₙNR^{c}R^{d},
(9) —C(O)R^{c},
(10) —CN,
(11) —C(O)NR^{c}R^{d},
(12) —NR^{c}C(O)R^{d},
(13) —CF₃,
(14) —OCF₃,
(15) C₃₋₈cycloalkyl, and
(16) cycloheteroalkyl;
each R^{b}is independently selected from:
(1) C₁₋₆alkyl,
(2) —OR^{c},
(3) halogen,
(4) —CN,
(5) —C(O)NR^{c}R^{d},
(6) —NR^{c}C(O)R^{d},
(7) CF₃,
(8) —OCF₃, and
(9) phenyl;
each R^{c} and R^{d} is independently selected from:
(1) hydrogen,
(2) —C₁₋₁₀alkyl,
(3) —NH(C₁₋₁₀alkyl),
(4) —N(C₁₋₁₀alkyl)₂,
(5) cycloalkyl, and
(6) cycloheteroalkyl, or
R^{c} and R^{d} together with the atom to which they are attached form a heterocyclic ring of 4 to 7 members containing 0-2 additional heteroatoms independently selected from oxygen, sulfur and N—R^{c}, each R^{c} and R^{d} may be unsubstituted or substituted with one to three substituents selected from R^{e};
each R^{e} and R^{f} is independently selected from:
(1) hydrogen,
(2) C₁₋₁₀alkyl,
(3) cycloheteroalkyl,
(4) cycloheteroalkyl-C₁₋₁₀alkyl,
(5) aryl,
(6) heteroaryl,
(7) aryl-C₁₋₁₀alkyl, and
(8) heteroaryl-C₁₋₁₀alkyl, or
R^{e} and R^{f} together with the carbon to which they are attached form a ring of 5 to 7 members containing 0-2 heteroatoms independently selected from oxygen, sulfur and nitrogen;
or a pharmaceutically acceptable salt thereof.

7. The method according to claim 6, wherein in the compound of structural formula I:
R¹ is selected from:
(1) hydrogen, and
(2) C₁₋₄alkyl;
R² is selected from:
(1) —OR^{c},
(2) —NR^{c}R^{d},
(3) —NR^{c}C(O)R^{d},
(4) —NR^{c}SO₂R^{d},
(5) —C(O)OR^{c}, and
(6) —C(O)NR^{c}R^{d};
R³ is selected from:
(1) —methyl, and
(2) —Ar²;
Ar¹ and Ar² are phenyl, each optionally substituted with one or two groups independently selected from R^{b};
each R^{e} and R^{f} is independently selected from:
(1) hydrogen,
(2) C₁₋₁₀alkyl,
(3) cycloheteroalkyl,
(4) aryl, and
(5) heteroaryl;
or a pharmaceutically acceptable salt thereof.

8. The method according to claim 7, wherein in the compound of structural formula I:
R¹ is selected from:
(1) hydrogen,
(2) methyl, and
(3) ethyl;
R² is selected from:
(1) —OR^{c},
(2) —NR^{c}C(O)R^{d},
(3) —C(O)OR^{c}, and
(4) —C(O)NR^{c}R^{d};
Ar¹ and Ar² are each independently selected from:
(1) phenyl,
(2) 4-fluorophenyl,
(3) 2-chlorophenyl,
(4) 3-chlorophenyl,
(5) 4-chlorophenyl,
(6) 4-cyanophenyl,
(7) 4-methylphenyl,
(8) 4-isopropylphenyl,
(9) 4-biphenyl,
(10) 4-bromophenyl,
(11) 4-iodophenyl,
(12) 2,4-dichlorophenyl, and
(13) 2-chloro4-fluorophenyl;
each R^{c} and R^{d} is independently selected from:
(1) hydrogen,
(2) methyl,
(3) ethyl,
(4) —N(CH₃)₂,
(5) —NH(CH₃),
(6) cyclopentane,
(7) cyclohexane,
(8) cycloheptane,
(9) piperidine,
(10) morpholine,
(11) pyrrolidine,
(12) azepine, and
(13) 4-methylpiperazine,
each R^{c} and R^{d} may be unsubstituted or substituted with one to three substituents selected from R^{e};
or a pharmaceutically acceptable salt thereof.

9. The method according to claim 8, wherein in the compound according to structural formula I:
R¹ is methyl;
R² is —C(O)NR^{c}R^{d};
R³ is —Ar²;
Ar¹ is 2,4-dichlorophenyl;
Ar² is 4-chlorophenyl;
each R^{c} and R^{d} is independently selected from:
(1) hydrogen,
(2) cyclohexane, and
(3) piperidine,
each R^{c} and R^{d} may be unsubstituted or substituted with one to three substituents selected from R^{e};
or a pharmaceutically acceptable salt thereof.

10. The method according to claim 1, wherein the compound of structural formula I is selected from:
(1) ethyl 1-(4-chlorophenyl)-2-(2,4-dichlorophenyl)-5-methyl-imidazole-4-carboxylate;
(2) ethyl 2-(2,4-dichlorophenyl)-1,5-dimethyl-imidazole-4-carboxylate;
(3) N-(cyclohexyl)-1-(4-chlorophenyl)-2-(2,4-dichlorophenyl)-5-methyl-imidazole-4-carboxamide;
(4) N-(piperidin-1-yl)-1-(4-chlorophenyl)-2-(2,4-dichlorophenyl)-5-methyl-imidazole-4-carboxamide;
(5) N-(piperidin-1-yl)-2-(2,4-dichlorophenyl)-1,5-dimethyl-imidazole-4-carboxamide;
(6) N-(cyclopentyl)-1-(4-chlorophenyl)-2-(2,4-dichlorophenyl)-5-methyl-imidazole-4-carboxamide;
(7) N-(cycloheptyl)-1-(4-chlorophenyl)-2-(2,4-dichlorophenyl)-5-methyl-imidazole-4-carboxamide;
(8) N-(morpholin4-yl)-1-(4-chlorophenyl)-2-(2,4-dichlorophenyl)-5-methyl-imidazole-4-carboxamide;
(9) N-(pyrrolidin-1-yl)-1-(4-chlorophenyl)-2-(2,4-dichlorophenyl)-5-methyl-imidazole-4-carboxamide;
(10) N-(azepin-1-yl)-1-(4-chlorophenyl)-2-(2,4-dichlorophenyl)-5-methyl-imidazole-4-carboxamide;
(11) N-(4-methylpiperazin-1-yl)-1-(4-chlorophenyl)-2-(2,4-dichlorophenyl)-5-methyl-imidazole-4-carboxamide;
(12) N′,N′-dimethyl-1-(4-chlorophenyl)-2-(2,4-dichlorophenyl)-5-methyl-imidazole-4-carboxhydrazide;
(13) N-(cyclohexyl)-1-(phenyl)-2-(2,4-dichlorophenyl)-5-methyl-imidazole-4-carboxamide;
(14) N-(piperidin-1-yl)-1-(phenyl)-2-(2,4-dichlorophenyl)-5-methyl-imidazole-4-carboxamide;
(15) N-(cyclohexyl)-1-(4-fluorophenyl)-2-(2,4-dichlorophenyl)-5-methyl-imidazole-4-carboxamide;
(16) N-(piperidin-1-yl)-1-(4-fluorophenyl)-2-(2,4-dichlorophenyl)-5-methyl-imidazole-4-carboxamide;
(17) N-(4-methyl-cyclohexyl)-1-(4-chlorophenyl)-2-(2,4-dichlorophenyl)-5-methyl-imidazole-4-carboxamide (Isomer A);
(18) N-(2-(pyrrolidin-1-yl)ethyl)-1-(4-chlorophenyl)-2-(2,4-dichlorophenyl)-5-methyl-imidazole-4-carboxamide;
(19) N-(cyclohexyl)-1-(4-chlorophenyl)-2-(2-chlorophenyl)-5-methyl-imidazole-4-carboxamide;
(20) N-(piperidin-1-yl)-1-(4-chlorophenyl)-2-(2-chlorophenyl)-5-methyl-imidazole-4-carboxamide;
(21) N-(cyclohexyl)-1-(4-bromophenyl)-2-(2,4-dichlorophenyl)-5-methyl-imidazole-4-carboxamide;
(22) N-(piperidin-1-yl)-1-(4-bromophenyl)-2-(2,4-dichlorophenyl)-5-methyl-imidazole-4-carboxamide;
(23) N-(cyclohexyl)-1-(4-isopropylphenyl)-2-(2,4-dichlorophenyl)-5-methyl-imidazole-4-carboxamide;
(24) N-(piperidin-1-yl)-1-(4-isopropylphenyl)-2-(2,4-dichlorophenyl)-5-methyl-imidazole-4-carboxamide;
(25) N-(cyclohexyl)-1-(4-cyanophenyl)-2-(2,4-dichlorophenyl)-5-methyl-imidazole-4-carboxamide;
(26) N-(piperidin-1-yl)-1-(4-cyanophenyl)-2-(2,4-dichlorophenyl)-5-methyl-imidazole-4-carboxamide;
(27) N-(cyclohexyl)-1-(4-biphenyl)-2-(2,4-dichlorophenyl)-5-methyl-imidazole-4-carboxamide;
(28) N-(piperidin-1-yl)-1-(4-bipheny1)-2-(2,4-dichlorophenyl)-5-methyl-imidazole-4-carboxamide;
(29) N-(cyclohexyl)-1,2-bis(4-chlorophenyl)-5-methyl-imnidazole-4-carboxamide;
(30) N-(piperidin-1yl)-1,2-bis(4-chlorophenyl)-5-methyl-imidazole-4-carboxamide;
(31) N-(cyclohexyl)-1-(4-chlorophenyl)-2-(2,4-dichlorophenyl)-5-ethyl-imidazole-4-carboxamide;
(32) N-(piperidin-1-yl)-1-(4-chlorophenyl)-2-(2,4-dichlorophenyl)-5-ethyl-imidazole-4-carboxamide;
(33) N-(cyclohexyl)-1-(4-bromophenyl)-2-(2,4-dichlorophenyl)-5-ethyl-imidazole-4-carboxamide;
(34) N-(piperidin-1-yl)-1-(4-bromophenyl)-2-(2,4-dichlorophenyl)-5-ethyl-imidazole-4-carboxamide;
(35) N-(cyclohexyl)-1-(4-isopropylphenyl)-2-(2,4-dichlorophenyl)-5-ethyl-imidazole-4-carboxamide;
(36) N-(piperidin-1-yl)-1-(4-isopropylphenyl)-2-(2,4-dichlorophenyl)-5-ethyl-imidazole-4-carboxamide;
(37) N-(cyclohexyl)-1-(3-chlorophenyl)-2-(2,4-dichlorophenyl)-5-methyl-imidazole-4-carboxamide;
(38) N-(cyclohexyl)-1-(4-chlorophenyl)-2-(2-chloro4-fluorophenyl)-5-methyl-imidazole-4-carboxamide;
(39) N-(piperidin-1-yl)-1-(4-chlorophenyl)-2-(2-chloro4-fluorophenyl)-5-methyl-imidazole-4-carboxamide;
(40) N-(cyclohexyl)-1-(2-chlorophenyl)-2-(2,4-dichlorophenyl)-5-methyl-imidazole-4-carboxamide; and
(41) N-(cyclohexyl)-1-(4-chlorophenyl)-2-(3-chlorophenyl)-5-methyl-imidazole-4-carboxamide;
or a pharmaceutically acceptable salt thereof.

11. The method according to claim 10, wherein the compound of structural formula I is selected from:
(1) N-(cyclohexyl)-1-(4-chlorophenyl)-2-(2,4-dichlorophenyl)-5-methyl-imidazole-4-carboxamide;
(2) N-(piperidin-1-yl)-1-(4-chlorophenyl)-2-(2,4-dichlorophenyl)-5-methyl-imidazole-4-carboxamide;
(3) N-(cyclohexyl)-1-(4-bromophenyl)-2-(2,4-dichlorophenyl)-5-ethyl-imidazole-4-carboxamide;
(4) N-(cyclohexyl)-1-(4-isopropylphenyl)-2-(2,4-dichlorophenyl)-5-ethyl-imidazole-4-carboxamide;
(5) N-(cyclohexyl)-1-(4-bromophenyl)-2-(2,4-dichlorophenyl)-5-methyl-imidazole-4-carboxamide; and
(6) N-(cycloheptyl)-1-(4-chlorophenyl)-2-(2,4-dichlorophenyl)-5-methyl-imidazole-4-carboxamide;
or a pharmaceutically acceptable salt thereof.

12. A method of preventing obesity in a person at risk for obesity comprising administration to said person of about 0.001 mg to about 100 mg per kg of a compound of structural formula I: 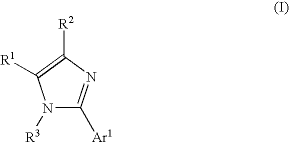
or a pharmaceutically acceptable salt thereof, wherein:
R¹ is selected from:
(1) hydrogen,
(2) C₁₋₄alkyl,
(3) C₂₋₄alkenyl,
(4) C₂₋₄alkynyl,
(5) C₃₋₇cycloalkyl,
(6) C₃₋₇cycloalkyl-C₁₋₄alkyl,
(7) cycloheteroalkyl,
(8) cycloheteroalkyl-C₁₋₄alkyl,
(9) aryl, and
(10) heteroaryl;
wherein alkyl, alkenyl, alkynyl, cycloalkyl, and cycloheteroalkyl are optionally substituted with one to four substituents independently selected from R^{a}, and aryl and heteroaryl are optionally substituted with one to four substituents independently selected from R^{b};
R² is selected from:
(1) —OR^{c},
(2) —OC(O)R^{c},
(3) —OC(O)NR^{c}R^{d},
(4) —SR^{c},
(5) —S(O)ₘR^{c},
(6) —SO₂NR^{CR}^{d},
(7) —NR^{c}R^{d},
(8) —NR^{c}C(O)R^{d},
(9) —NR^{c}SO₂R^{d},
(10) —NR^{c}C(O)NR^{CR}^{d},
(11) —NR^{c}C(O)OR^{d},
(12) —C(O)OR^{c}, and
(13) —C(O)NR^{c}R^{d};
R³ is selected from:
(1) —C₁₋₁₀alkyl, and
(2) —Ar²;
Ar¹ and Ar² are independently selected from phenyl, naphthyl, thienyl, furanyl, pyrrolyl, benzothienyl, benzofuranyl, indanyl, indenyl, indolyl, tetrahydronaphthyl, 2,3-dihydrobenzofuranyl, dihydrobenzopyranyl, and 1,4-benzodioxanyl, each optionally substituted with one, two, or three groups independently selected from R^{b};
each R^{a} is independently selected from:
(1) —OR^{c},
(2) —NR^{c}S(O)ₘR^{d},
(3) halogen,
(4) —S(O)ₘR^{c},
(5) —SR^{c},
(6) —S(O)₂OR^{c},
(7) —S(O)ₘNR^{c}R^{d},
(8) —NR^{c}R^{d},
(9) —O(CR^{e}R^{f})ₙNR^{c}R^{d},
(10) —C(O)R^{c},
(1 1) —CO₂R^{c},
(12) —CO₂(CR^{e}R^{f})ₙCONR^{c}R^{d},
(13) —OC(O)R^{c},
(14) —CN,
(15) —C(O)NR^{c}R^{d},
(16) —NR^{c}C(O)R^{d},
(17) —OC(O)NR^{c}R^{d},
(18) —NR^{c}C(O)OR^{d},
(19) —NR^{c}C(O)NR^{c}R^{d},
(20) —CR^{c}(N—OR^{d}),
(21) —CF₃,
(22) —OCF₃,
(23) C₃₋₈cycloalkyl, and
(24) cycloheteroalkyl;
each R^{b} is independently selected from:
(1) C₁₋₆alkyl,
(2) C₂₋₆alkenyl,
(3) C₂₋₆alkynyl,
(4) —OR^{c},
(5) —NR^{c}S(O)ₘR^{d},
(6) —NO₂,
(7) halogen,
(8) —S(O)ₘR^{c},
(9) —SR^{c},
(10) —S(O)₂OR^{c},
(11) —S(O)ₘNR^{c}R^{d},
(12) —NR^{c}R^{d},
(13) —O(CR^{e}R^{f})ₙNR^{c}R^{d},
(14) —C(O)R^{c},
(15) —CO₂R^{c},
(16) —CO₂(CR^{e}R^{f})ₙCONR^{c}R^{d},
(17) —OC(O)R^{c},
(18) —CN,
(19) —C(O)NR^{c}R^{d},
(20) —NR^{c}C(O)R^{d},
(21) —OC(O)NR^{c}R^{d},
(22) —NR^{c}C(O)OR^{d},
(23) —NR^{c}C(O)NR^{c}R^{d},
(24) —CR^{c}(N—OR^{d}),
(25) —CF₃,
(26) —OCF₃,
(27) C₃₋₈cycloalkyl,
(28) cycloheteroalkyl, and
(29) phenyl;
each R^{c} and R^{d} is independently selected from:
(1) hydrogen,
(2) C₁₋₁₀alkyl,
(3) C₂₋₁₀alkenyl,
(4) C₂₋₁₀alkynyl,
(5) —N(C₁₋₁₀alkyl)₂,
(6) —NH(C₁₋₁₀alkyl),
(7) cycloalkyl,
(8) cycloalkyl-C₁₋₁₀alkyl;
(9) cycloheteroalkyl,
(10) cycloheteroalkyl-C₁₋₁₀alkyl;
(11) aryl,
(12) heteroaryl,
(13) aryl-C₁₋₁₀alkyl, and
(14) heteroaryl-C₁₋₁₀alkyl, or
R^{c} and R^{d} together with the atom to which they are attached form a heterocyclic ring of 4 to 7 members containing 0-2 additional heteroatoms independently selected from oxygen, sulfur and N—R^{c}, each R^{c} and R^{d} may be unsubstituted or substituted with one to three substituents selected from R^{e}; each R^{e} and R^{f} is independently selected from:
(1) hydrogen,
(2) C₁₋₁₀alkyl,
(3) C₂₋₁₀alkenyl,
(4) C₂₋₁₀alkynyl,
(5) cycloalkyl,
(6) cycloalkyl-C₁₋₁₀alkyl,
(7) cycloheteroalkyl,
(8) cycloheteroalkyl-C₁₋₁₀alkyl,
(9) aryl,
(10) heteroaryl,
(11) aryl-C₁₋₁₀alkyl, and
(12) heteroaryl-C₁₋₁₀alkyl, or
R^{e} and R^{f} together with the carbon to which they are attached form a ring of 5 to 7 members containing 0-2 heteroatoms independently selected from oxygen, sulfur and nitrogen;
m is selected from 1 and 2; and
n is selected from 1, 2, and 3.

13. A compound of structural formula I: 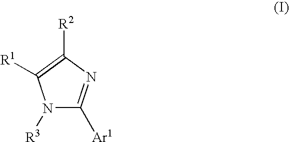
or a pharmaceutically acceptable salt thereof, wherein:
R¹ is selected from: methyl and ethyl,
wherein methyl and ethyl are optionally substituted with one to four substituents independently selected from R^{a};
R² is selected from:
(1) —C(O)OR^{c}, and
(2) —C(O)NR^{c}R^{d};
R³ is selected from methyl and Ar²;
Ar¹ and Ar² are phenyl, each optionally substituted with one, two, or three groups independently selected from R^{b};
each R^{a} is independently selected from:
(1) —OR^{c},
(2) —NR^{c}S(O)ₘR^{d},
(3) halogen,
(4) —S(O)ₘR^{c},
(5) —SR^{c},
(6) —S(O)ₘNR^{c}R^{d},
(7) —NR^{c}R^{d},
(8) —O(CR^{e}R^{f})ₙNR^{c}R^{d},
(9) —C(O)R^{c},
(10) —CN,
(11) —C(O)NR^{c}R^{d},
(12) —NR^{c}C(O)R^{d},
(13) —CF₃, and
(14) —OCF₃;
each R^{b} is independently selected from: halogen, C₁₋₃alkyl, —CN, and phenyl;
each R^{c} and R^{d} is independently selected from:
(1) hydrogen,
(2) C₁₋₁₀alkyl,
(3) C₂₋₁₀alkenyl,
(4) C₂₋₁₀alkynyl,
(5) —NH(C₁₋₁₀alkyl),
(6) —N(C₁₋₁₀alkyl)₂,
(7) cycloalkyl,
(8) cycloalkyl-C₁₋₁₀alkyl,
(9) cycloheteroalkyl,
(10) cycloheteroalkyl-C₁₋₁₀alkyl,
(11) aryl,
(12) heteroaryl,
(13) aryl-C₁₋₁₀alkyl, and
(14) heteroaryl-C₁₋₁₀alkyl, or
R^{c} and R^{d} together with the atom to which they are attached form a heterocyclic ring of 4 to 7 members containing 0-2 additional heteroatoms independently selected from oxygen, sulfur and N—R^{c}, each R^{c} and R^{d} may be unsubstituted or substituted with one to three substituents selected from R^{e};
each R^{e} and R^{f} is independently selected from:
(1) hydrogen,
(2) C₁₋₁₀alkyl,
(3) C₂₋₁₀alkenyl,
(4) C₂₋₁₀alkynyl,
(5) cycloalkyl,
(6) cycloalkyl-C₁₋₁₀alkyl,
(7) cycloheteroalkyl,
(8) cycloheteroalkyl-C₁₋₁₀alkyl,
(9) aryl,
(10) heteroaryl,
(11) aryl-C₁₋₁₀alkyl, and
(12) heteroaryl-C₁₋₁₀alkyl, or
R^{e} and R^{f} together with the carbon to which they are attached form a ring of 5 to 7 members containing 0-2 heteroatoms independently selected from oxygen, sulfur and nitrogen;
m is selected from 1 and 2; and
n is selected from 1, 2, and 3.

14. The compound of claim 13 wherein:
R¹ is methyl;
R² is —C(O)NR^{c}R^{d};
R³ is selected from methyl and Ar²;
Ar¹ is phenyl substituted at the 2 and 4 positions with a substituent independently selected from R^{b};
Ar² is independently selected from:
(1) phenyl,
(2) 4-fluorophenyl,
(3) 2-chlorophenyl,
(4) 3-chlorophenyl,
(5) 4-chlorophenyl,
(6) 4-cyanophenyl,
(7) 4-methylphenyl,
(8) 4-isopropylphenyl,
(9) 4-biphenyl,
(10) 4-bromophenyl,
(11) 4-iodophenyl,
(12) 2,4-dichlorophenyl, and
(13) 2-chloro-4-fluorophenyl;
each R^{c} and R^{d} is independently selected from:
(1) hydrogen,
(2) methyl,
(3) ethyl,
(4) —N(CH₃)₂,
(5) —NH(CH₃),
(6) cyclopentane,
(7) cyclohexane,
(8) cycloheptane,
(9) piperidine
(10) morpholine,
(11) pyrrolidine,
(12) azepine, and
(13) 4-methylpiperazine,
each R^{c} and R^{d} may be unsubstituted or substituted with one to three substituents selected from R^{e};
or a pharmaceutically acceptable salt thereof.

15. A compound selected from:
(1) ethyl 1-(4-chlorophenyl)-2-(2,4-dichlorophenyl)-5-methyl-imidazole-4-carboxylate;
(2) ethyl 2-(2,4-dichlorophenyl)-1,5-dimethyl-imidazole-4-carboxylate;
(3) N-(cyclohexyl)-1-(4-chlorophenyl)-2-(2,4-dichlorophenyl)-5-methyl-imidazole-4-carboxamide;
(4) N-(piperidin-1-yl)-1-(4-chlorophenyl)-2-(2,4-dichlorophenyl)-5-methyl-imidazole-4-carboxamide;
(5) N-(piperidin-1-yl)-2-(2,4chlorophenyl)-1,5-dimethyl-imidazole-4-carboxamide;
(6) N-(cyclopentyl)-1-(4-chlorophenyl)-2-(2,4-dichlorophenyl)-5-methyl-imidazole-4-carboxamide;
(7) N-(cycloheptyl)-1-(4-chlorophenyl)-2-(2,4-dichlorophenyl)-5-methyl-imidazole-4-carboxamide;
(8) N-(morpholin4-yl)-1-(4-chlorophenyl)-2-(2,4-dichlorophenyl)-5-methyl-imidazole-4-carboxamide;
(9) N-(pyrrolidin-1-yl)-1-(4-chlorophenyl)-2-(2,4-dichlorophenyl)-5-methyl-imidazole-4-carboxamide;
(10) N-(azepin-1-yl)-1-(4-chlorophenyl)-2-(2,4-dichlorophenyl)-5-methyl-imidazole-4-carboxamide;
(11) N-(4-methylpiperazin-1-yl)-1-(4-chlorophenyl)-2-(2,4-dichlorophenyl)-5-methyl-imidazole-4-carboxamide;
(12) N′,N′-dimethyl-1-(4-chlorophenyl)-2-(2,4-dichlorophenyl)-5-methyl-imidazole-4-carboxhydrazide;
(13) N-(cyclohexyl)-1-(phenyl)-2-(2,4-dichlorophenyl)-5-methyl-imidazole-4-carboxamide;
(14) N-(piperidin-1-yl)-1-(phenyl)-2-(2,4-dichlorophenyl)-5-methyl-imidazole-4-carboxamide;
(15) N-(cyclohexyl)-1-(4-fluorophenyl)-2-(2,4-dichlorophenyl)-5-methyl-imidazole-4-carboxamide;
(16) N-(piperidin-1-yl)-1-(4-fluorophenyl)-2-(2,4-dichlorophenyl)-5-methyl-imidazole-4-carboxamide;
(17) N-(4-methyl-cyclohexyl)-1-(4-chlorophenyl)-2-(2,4-dichlorophenyl)-5-methyl-imidazole-4-carboxamide (Isomer A);
(18) N-(2-(pyrrolidin-1-yl)ethyl)-1-(4-chlorophenyl)-2-(2,4-dichlorophenyl)-5-methyl-imidazole-4-carboxamide;
(19) N-(cyclohexyl)-1-(4-chlorophenyl)-2-(2-chlorophenyl)-5-methyl-imidazole-4-carboxamide;
(20) N-(piperidin-1-yl)-1-(4-chlorophenyl)-2-(2-chlorophenyl)-5-methyl-imidazole-4-carboxamide;
(21) N-(cyclohexyl)-1-(4-bromophenyl)-2-(2,4-dichlorophenyl)-5-methyl-imidazole-4-carboxamide;
(22) N-(piperidin-1-yl)-1-(4-bromophenyl)-2-(2,4-dichlorophenyl)-5-methyl-imidazole-4-carboxamide;
(23) N-(cyclohexyl)-1-(4-isopropylphenyl)-2-(2,4-dichlorophenyl)-5-methyl-imidazole-4-carboxamide;
(24) N-(piperidin-1-yl)-1-(4-isopropylphenyl)-2-(2,4-dichlorophenyl)-5-methyl-imidazole-4-carboxamide;
(25) N-(cyclohexyl)-1-(4-cyanophenyl)-2-(2,4-dichlorophenyl)-5-methyl-imidazole-4-carboxamide;
(26) N-(piperidin-1-yl)-1-(4-cyanophenyl)-2-(2,4-dichlorophenyl)-5-methyl-imidazole-4-carboxamide;
(27) N-(cyclohexyl)-1-(4-biphenyl)-2-(2,4-dichlorophenyl)-5-methyl-imidazole-4-carboxamide;
(28) N-(piperidin-1-yl)-1-(4-biphenyl)-2-(2,4-dichlorophenyl)-5-methyl-imidazole-4-carboxamide;
(29) N-(cyclohexyl)-1,2-bis(4-chlorophenyl)-5-methyl-imidazole-4-carboxamide;
(30) N-(piperidin-1-yl)-1,2-bis(4-chlorophenyl)-5-methyl-imidazole-4-carboxamide;
(31) N-(cyclohexyl)-1-(4-chlorophenyl)-2-(2,4-dichlorophenyl)-5-ethyl-imidazole-4-carboxamide;
(32) N-(piperidin-1-yl)-1-(4-chlorophenyl)-2-(2,4-dichlorophenyl)-5-ethyl-imidazole-4-carboxamide;
(33) N-(cyclohexyl)-1-(4-bromophenyl)-2-(2,4-dichlorophenyl)-5-ethyl-imidazole-4-carboxamide;
(34) N-(piperidin-1-yl)-1-(4-bromophenyl)-2-(2,4-dichlorophenyl)-5-ethyl-imidazole-4-carboxamide;
(35) N-(cyclohexyl)-1-(4-isopropylphenyl)-2-(2;4-dichlorophenyl)-5-ethyl-imidazole-4-carboxamide;
(36) N-(piperidin-1-yl)-1-(4-isopropylphenyl)-2-(2,4-dichlorophenyl)-5-ethyl-imidazole-4-carboxamide;
(37) N-(cyclohexyl)-1-(3-chlorophenyl)-2-(2,4-dichlorophenyl)-5-methyl-imidazole-4-carboxamide;
(38) N-(cyclohexyl)-1-(4-chlorophenyl)-2-(2-chloro4-fluorophenyl)-5-methyl-imidazole-4-carboxamide;
(39) N-(piperidin-1-yl)-1-(4-chlorophenyl)-2-(2-chloro4-fluorophenyl)-5-methyl-imidazole-4-carboxamide;
(40) N-(cyclohexyl)-1-(2-chlorophenyl)-2-(2,4-dichlorophenyl)-5-methyl-imidazole-4-carboxamide; and
(41) N-(cyclohexyl)-1-(4-chlorophenyl)-2-(3-chlorophenyl)-5-methyl-imidazole-4-carboxamide;
or a pharmaceutically acceptable salt thereof.

16. The compound according to claim 15 selected from:
(1) N-(cyclohexyl)-1-(4-chlorophenyl)-2-(2,4-dichlorophenyl)-5-methyl-imidazole-4-carboxamide;
(2) N-(piperidin-1-yl)-1-(4-chlorophenyl)-2-(2,4-dichlorophenyl)-5-methyl-imidazole-4-carboxamide;
(3) N-(cyclohexyl)-1-(4-bromophenyl)-2-(2,4-dichlorophenyl)-5-ethyl-imidazole-4-carboxamide;
(4) N-(cyclohexyl)-1-(4-isopropylphenyl)-2-(2,4-dichlorophenyl)-5-ethyl-imidazole-4-carboxamide;
(5) N-(cyclohexyl)-1-(4-bromophenyl)-2-(2,4-dichlorophenyl)-5-methyl-imidazole-4-carboxamide; and
(6) N-(cycloheptyl)-1-(4-chlorophenyl)-2-(2,4-dichlorophenyl)-5-methyl-imidazole-4-carboxamide; or a pharmaceutically acceptable salt thereof.

17. A composition comprising a compound according to claim 13 and a pharmaceutically acceptable carrier.

18. A composition comprising a compound according to claim 15 and a pharmaceutically acceptable carrier.

19. A method of preventing obesity in a person at risk for obesity comprising administration to said person of about 0.001 to about 100 mg/kg of a compound according to claim 13.

20. A method of preventing obesity in a person at risk for obesity comprising administration to said person of about 0.001 to about 100 mg/kg of a compound according to claim 15.

21. A composition comprising a pharmaceutically effective amount of a compound of structural formula I: 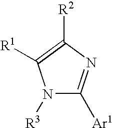
or a pharmaceutically acceptable salt thereof, wherein:
R¹ is selected from:
(1) hydrogen,
(2) C₁₋₄alkyl,
(3) C₂₋₄alkenyl,
(4) C₂₋₄alkynyl,
(5) C₃₋₇cycloalkyl,
(6) C₃₋₇cycloalkyl-C₁₋₄alkyl,
(7) cycloheteroalkyl,
(8) cycloheteroalkyl-C₁₋₄alkyl,
(9) aryl, and
(10) heteroaryl;
wherein alkyl, alkenyl, alkynyl, cycloalkyl, and cycloheteroalkyl are optionally substituted with one to four substituents independently selected from R^{a}, and aryl and heteroaryl are optionally substituted with one to four substituents independently selected from R^{b};
R² is selected from:
(1) —OR^{c},
(2) —OC(O)R^{c},
(3) —OC(O)NR^{c}R^{d},
(4) —SR^{c},
(5) —S(O)ₘR^{c},
(6) —SO₂NR^{c}R^{d},
(7) —NR^{c}R^{d},
(8) —NR^{c}C(O)R^{d},
(9) —NR^{c}SO₂R^{d},
(10) —NR^{c}C(O)NR^{c}R^{d},
(11) —NR^{c}C(O)OR^{d},
(12) —C(O)OR^{c}, and
(13) —C(O)NR^{c}R^{d};
R³ is selected from:
(1) —C₁₋₁₀alkyl, and
(2) —Ar₂;
Ar¹ and Ar² are independently selected from phenyl, naphthyl, thienyl, furanyl, pyrrolyl, benzothienyl, benzofuranyl, indanyl, indenyl, indolyl, tetrahydronaphthyl, 2,3-dihydrobenzofuranyl, dihydrobenzopyranyl, and 1,4-benzodioxanyl, each optionally substituted with one, two, or three groups independently selected from R^{b};
each R^{a} is independently selected from:
(1) —OR^{c},
(2) —NR^{c}S(O)ₘR^{d},
(3) halogen,
(4) —S(O)ₘR^{c},
(5) —SR^{c},
(6) —S(O)₂OR^{c},
(7) —S(O)ₘNR^{c}R^{d},
(8) —NR^{c}R^{d},
(9) —O(CR^{e}R^{f})ₙNR^{c}R^{d},
(10) —C(O)R^{c},
(11) —CO₂R^{c},
(12) —CO₂(CR^{e}R^{f})ₙCONR^{c}R^{d},
(13) —OC(O)R^{c},
(14) —CN,
(15) —C(O)NR^{c}R^{d},
(16) —NR^{c}C(O)R^{d},
(17) —OC(O)NR^{c}R^{d},
(18) —NR^{c}C(O)OR^{d},
(19) —NR^{c}C(O)NR^{c}R^{d},
(20) —CR^{c}(N—OR^{d}),
(21) —CF₃,
(22) —OCF₃,
(23) —C₃₋₈cycloalkyl, and
(24) cycloheteroalkyl;
each R^{b} is independently selected from:
(1) C₁₋₆alkyl,
(2) C₂₋₆alkenyl,
(3) C₂₋₆alkynyl,
(4) —OR^{c},
(5) —NR^{c}S(O)ₘR^{d},
(6) —NO₂,
(7) halogen,
(8) —S(O)ₘR^{c},
(9) —SR^{c},
(10) —S(O)₂OR^{c},
(11) —S(O)ₘNR^{c}R^{d},
(12) —NR^{c}R^{d},
(13) —O(CR^{e}R^{f})ₙNR^{c}R^{d},
(14) —C(O)R^{c},
(15) —CO₂R^{c},
(16) —CO₂(CR^{e}R^{f})ₙCONR^{c}R^{d},
(17) —OC(O)R^{c},
(18) —CN,
(19) —C(O)NR^{c}R^{d},
(20) —NR^{c}C(O)R^{d},
(21) —OC(O)NR^{c}R^{d},
(22) —NR^{c}C(O)OR^{d},
(23) —NR^{c}C(O)NR^{c}R^{d},
(24) —CR^{c}(N—OR^{d}),
(25) —CF₃,
(26) —OCF₃,
(27) C₃₋₈cycloalkyl,
(28) cycloheteroalkyl, and
(29) phenyl;
each R^{c} and R^{d} is independently selected from:
(1) hydrogen,
(2) C₁₋₁₀alkyl,
(3) C₂₋₁₀alkenyl,
(4) C₂₋₁₀alkynyl,
(5) —NH(C₁₋₁₀alkyl),
(6) —N(C₁₋₁₀alkyl)₂,
(7) cycloalkyl,
(8) cycloalkyl-C₁₋₁₀alkyl;
(9) cycloheteroalkyl,
(10) cycloheteroalkyl-C₁₋₁₀alkyl;
(11) aryl,
(12) heteroaryl,
(13) aryl-C₁₋₁₀alkyl, and
(14) heteroaryl-C₁₋₁₀alkyl, or
R^{c} and R^{d} together with the atom to which they are attached form a heterocyclic ring of 4 to 7 members containing 0-2 additional heteroatoms independently selected from oxygen, sulfur and N—R^{c}, each R^{c} and R^{d} may be unsubstituted or substituted with one to three substituents selected from R^{e};
each R^{e} and R^{f} is independently selected from:
(1) hydrogen,
(2) C₁₋₁₀alkyl,
(3) C₂₋₁₀alkenyl,
(4) C₂₋₁₀alkynyl,
(5) cycloalkyl,
(6) cycloalkyl-C₁₋₁₀alkyl,
(7) cycloheteroalkyl,
(8) cycloheteroalkyl-C₁₋₁₀alkyl,
(9) aryl,
(10) heteroaryl,
(11) aryl-C₁₋₁₀alkyl, and
(12) heteroaryl-C₁₋₁₀alkyl, or
R^{e} and R^{f} together with the carbon to which they are attached form a ring of 5 to 7 members containing 0-2 heteroatoms independently selected from oxygen, sulfur and nitrogen;
m is selected from 1 and 2; and
n is selected from 1, 2, and 3;
and an anorectic agent selected from: aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; or
a selective serotonin reuptake inhibitor selected from: fluoxetine, fluvoxamine, paroxetine and sertraline; or an antidepressant agent selected from: norepinephrine reuptake inhibitors, selective serotonin reuptake inhibitors, monoamine oxidase inhibitors, reversible inhibitors of monoamine oxidase, serotonin and noradrenaline reuptake inhibitors, corticotropin releasing factor antagonists, α-adrenoreceptor antagonists and atypical anti-depressants; or
the VLA-4 antagonist natalizumab; or a steroid or corticosteroid selected from: beclomethasone, methylprednisolone, betamethasone, prednisone, dexamethasone, and hydrocortisone; or an antihistamine selected from: bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, and descarboethoxyloratadine; or a non-steroidal anti-asthmatics selected from: theophylline, cromolyn sodium, atropine, and ipratropium bromide; or a β2-agonist selected from: terbutaline, metaproterenol, fenoterol, isoetharine, albuterol, bitolterol, salmeterol, epinephrine, and pirbuterol; or a leukotriene antagonist selected from: zafirlukast, montelukast, pranlukast, iralukast, pobilukast, and SKB-106,203; or a leukotriene biosynthesis inhibitors selected from: zileuton, and BAY-1005; or an anti-cholinergic agent selected from ipratropium bromide and atropine: or an antagonist of the CCR-3 chemokine receptors, or
an osmotic agent selected from sorbitol, lactulose, polyethylene glycol, magnesium, phosphate,and sulfate; or a laxative selected from: magnesium and docusate sodium; or a bulking agent selected from: psyllium, methylcellulose, and calcium polycarbophil; or a stimulant selected from an anthroquinone, and phenolphthalein; or
a corticosteroid; or penicillamine, or colchicine; or an interferon-γ, 2-oxoglutarate analog; or a prostaglandin analog; or an anti-inflammatory drug selected from: azathioprine, methotrexate, leflunamide, indomethacin, and naproxen;
and a pharmaceutically acceptable carrier.

22.-31. (Cancelled)
